(19) United States
(12) Patent Application Publication
(10) Pub. No.: US 2014/0248361 A1
(43) Pub. Date: Sep. 4, 2014

(54) HERBAL EXTRACT COMPOSITION AND A PROCESS THEREOF

(71) Applicant: PRASAN SOLUTIONS (INDIA) PVT LTD, Kerala, Ernakulam District (IN)

(72) Inventors: Sanjith Mooppil, Malappuram (IN); Pramod Francis, Ernakulam (IN)

(21) Appl. No.: 14/348,667

(22) PCT Filed: Aug. 14, 2013
(86) PCT No.: PCT/IB2013/056643

§ 371 Date: Mar. 31, 2014

(30) Foreign Application Priority Data
Aug. 14, 2012 (IN) 3360/CHE/2012

(51) Int. Cl. A23D 9/06; A61K 8/02; C11B 5/00; A61Q 19/00; B02C 19/18; A61K 8/97; A61K 8/34

(52) U.S. Cl. CPC A23D 9/06; A61K 8/97; A61K 8/02; A61K 8/345; A61Q 19/00; B02C 19/186; C11B 5/0085
USPC 424/489; 514/783; 241/30; 241/23; 252/398; 554/2

(57) ABSTRACT

A composition is provided that includes extracts of green tea and rosemary extract optionally along with adjuvant and/or excipient. In addition, provided is a process for the preparation of said composition. Also provided is a composition including extract of green tea, rosemary extract and synthetic antioxidant optionally along with adjuvant and/or excipient.

### TECHNICAL FIELD

The present invention relates to a composition comprising extracts of green tea and rosemary extract optionally along with adjuvant and/or excipient. In addition, the present invention relates to a process for the preparation of said composition. The instant invention further relates to a composition comprising extract of green tea, rosemary extract and synthetic antioxidant optionally along with adjuvant and/or excipient.

### BACKGROUND AND PRIOR ART OF THE INVENTION

Currently, food preservation systems often use chemicals and heat treatments to reduce the risk of bacterial food poisoning outbreaks and food spoilage, but chemicals can alter the taste of the product and, moreover, can compromise food safety.

In the past several years, a lot of work has been done in developing products that would serve as preservatives that could prevent the oxidation of fats, vegetable oils, carotenoids and their biologically active derivatives such as essential oils and other flavoring products so that the degradation of their quality is prevented in foodstuffs. Fats, oils and their products become rancid or unpleasant by odor or flavor due to the oxidative effects. The prior art shows various methods of inhibiting oxidation by addition of fat soluble antioxidants to the foodstuff. A number of chemical compounds are employed for avoiding or reducing these effects, so that, fats and oils or food containing fats and oils can be kept for longer periods of time. However, such agents have not been satisfactorily effective in many ways.

The chemical anti-oxidants that had been made use of in such cases include BHA (butylated hydroxyanisole), BHT (butylated hydroxyl toluene) and TBHQ (tertiary butyl hydroquinone) as well as other chemicals such as propyl gallate (PG). However, their volatility and tendency to decompose at higher temperature make them less suitable for deep fat or oil fried foods. Also, they were not found to be effective in protecting certain off-flavor development or the so called reversion flavor that occurs with the passage of time in oils like soybean oil.

It has been found that certain plant materials or extracts such as grape seed extracts, green tea extracts, sage, clove bud oil, clove leaf oil, Vitamin C, cinnamon leaf oil, oleoresin turmeric, tocopherol, tocotrienol, rosemary extracts and gallic acid etc., or salts thereof, were having anti-oxidant properties and their use as anti-oxidant preservatives have been widely discussed in past two decades. The use of natural anti-oxidants such as green tea as stabilizers for fats, oils, fatty food and ingredients of food is also discussed widely in U.S. Pat. No. 3,812,266 and U.S. Pat. No. 3,451,832. These patents describe the use of green tea and the importance of green tea catachin in the anti-oxidant activity of green tea. The work done in U.S. Pat. No. 4,840,966 and CA Patent No. 1057113A1 shows the health benefits of green tea and other such natural anti-oxidants.

Green tea is made from unfermented leaves and reportedly contains the highest concentration of powerful antioxidants called polyphenols, high antioxidant activity of green tea extracts, are used as a kind of innovative food additive to preserve pork, chicken meat, vegetable oil, fish oil and fish flesh, food emulsions and animal fat. Even though a number of antioxidants and various combinations thereof have been disclosed in the various inventions, there is still a need for additional antioxidant compositions in its right formulator form having improved characteristics.

With all the health benefits and the advantages of being all natural products, the use of such natural extracts needed further development of processes of extraction and the development of a right formulator form thereby making them effectively usable in such applications. The solubility factor is most important in such cases, where the formulated product had to be oil and fat soluble for them to be effective in their action as antioxidants.

In recent past several products are formulated for various application in fats and oil applications. Though the products developed from these natural products so far were found to be effective in their use as anti-oxidants, their use was not found satisfactory in terms of solubility factor with respect to time, sedimentation and extended shelf life in its use as preservatives in oils and fats and in processed foods such as deep fried foodstuffs. Thus, there was a need to develop a composition that can successfully overcome the difficulties stated above.

### SUMMARY OF THE INVENTION

Accordingly the present disclosure relates to a composition comprising green tea extract and rosemary extract, optionally along with adjuvant or excipient or a combination thereof; a process of preparing the composition comprising green tea extract and rosemary extract, said process comprising acts of: a) granulating crude extract of green tea and mixing the granulated extract with the rosemary extract to obtain a mixture, b) optionally adding adjuvant or excipient or a combination thereof to the mixture, and c) passing the mixture of step a) or b) through homogenizer to obtain said composition comprising green tea extract and rosemary extract; a composition comprising the composition as mentioned above and tertiary butyl hydroquinone; an oil, fat or cosmetic preparation, comprising the composition as mentioned above; and a method of preparing the oil, fat or cosmetic preparation as mentioned above, wherein said method comprises act of mixing the composition as mentioned above with the oil, fat or cosmetic ingredients.

### BRIEF DESCRIPTION OF THE ACCOMPANYING FIGURES

FIG. 1A illustrates Oxidative stability of palm oil at temperatures 100° C., 120° C. and 140° C. upon addition of instant composition, sample A, sample B, sample C, sample D, sample E and sample F, respectively.

FIG. 1B illustrates increase in the protection factor of palm oil at 100° C., upon addition of instant composition, sample A, sample B, sample C, sample D, sample E and sample F, respectively.

FIG. 1C illustrates increase in the protection factor of palm oil at 120° C., upon addition of instant composition, sample A, sample B, sample C, sample D, sample E and sample F, respectively.

FIG. 1D illustrates increase in the protection factor of palm oil at 140° C., upon addition of instant composition, sample A, sample B, sample C, sample D, sample E and sample F, respectively.

FIG. 2A illustrates Oxidative stability of sun flower oil at temperatures 80° C., 100° C. and 120° C. upon addition of instant composition, sample A, sample B, sample C, sample D, sample E and sample F, respectively.

FIG. 2B illustrates increase in the protection factor of sun flower oil at 80° C., upon addition of instant composition, sample A, sample B, sample C, sample D, sample E and sample F, respectively.

FIG. 2C illustrates increase in the protection factor of sun flower oil at 100° C., upon addition of instant composition, sample A, sample B, sample C, sample D, sample E and sample F, respectively.

FIG. 2D illustrates increase in the protection factor of sun flower oil at 120° C., upon addition of instant composition, sample A, sample B, sample C, sample D, sample E and sample F, respectively.

FIG. 3A illustrates Oxidative stability of almond oil at temperatures 100° C., 120° C. and 140° C. upon addition of instant composition, sample B, sample B1 and sample B2, respectively.

FIG. 3B illustrates increase in the protection factor of almond oil at 100° C., upon addition of instant composition, sample B, sample B1 and sample B2, respectively.

FIG. 3C illustrates increase in the protection factor of almond oil at 120° C., upon addition of instant composition, sample B, sample B1 and sample B2, respectively.

FIG. 3D illustrates increase in the protection factor of almond oil at 140° C., upon addition of instant composition, sample B, sample B1 and sample B2, respectively.

FIG. 4A illustrates Oxidative stability of apricot oil at temperatures 100° C., 120° C. and 140° C. upon addition of instant composition, sample B, sample B1 and sample B2, respectively.

FIG. 4B illustrates increase in the protection factor of apricot oil at 100° C., upon addition of instant composition, sample B, sample B1 and sample B2, respectively.

FIG. 4C illustrates increase in the protection factor of apricot oil at 120° C., upon addition of instant composition, sample B, sample B1 and sample B2, respectively.

FIG. 4D illustrates increase in the protection factor of apricot oil at 140° C., upon addition of instant composition, sample B, sample B1 and sample B2, respectively.

FIG. 5A illustrates Oxidative stability of fish oil at temperatures 60° C., 70° C. and 80° C. upon addition of instant composition, sample B2, sample C and sample D, respectively.

FIG. 5B illustrates increase in the protection factor of fish oil at 60° C., upon addition of instant composition, sample B2, sample C and sample D, respectively.

FIG. 5C illustrates increase in the protection factor of fish oil at 70° C., upon addition of instant composition, sample B2, sample C and sample D, respectively.

FIG. 6A illustrates peroxide value of palm oil at ambient temperature, upon addition of instant composition, sample A, sample B, sample C, sample D, sample E and sample F, respectively.

FIG. 6B illustrates peroxide value of palm oil at 50° C., upon addition of instant composition, sample A, sample B, sample C, sample D, sample E and sample F, respectively.

FIG. 6C illustrates para-anisidine value of palm oil at ambient temperature, upon addition of instant composition, sample A, sample B, sample C, sample D, sample E and sample F, respectively.

FIG. 6D illustrates para-anisidine value of palm oil at 50° C., upon addition of instant composition, sample A, sample B, sample C, sample D, sample E and sample F, respectively.

FIG. 6E illustrates totox value of palm oil at ambient temperature, upon addition of instant composition, sample A, sample B, sample C, sample D, sample E and sample F, respectively.

FIG. 6F illustrates totox value of palm oil at 50° C., upon addition of instant composition, sample A, sample B, sample C, sample D, sample E and sample F, respectively.

FIG. 7A illustrates peroxide value of sunflower oil at ambient temperature, upon addition of instant composition, sample A, sample B, sample C, sample D, sample E and sample F, respectively.

FIG. 7B illustrates peroxide value of sunflower oil at 50° C., upon addition of instant composition, sample A, sample B, sample C, sample D, sample E and sample F, respectively.

FIG. 7C illustrates para-anisidine value of sunflower oil at ambient temperature, upon addition of instant composition, sample A, sample B, sample C, sample D, sample E and sample F, respectively.

FIG. 7D illustrates para-anisidine value of sunflower oil at 50° C., upon addition of instant composition, sample A, sample B, sample C, sample D, sample E and sample F, respectively.

FIG. 7E illustrates totox value of sunflower oil at ambient temperature, upon addition of instant composition, sample A, sample B, sample C, sample D, sample E and sample F, respectively.

FIG. 7F illustrates totox value of sunflower oil at 50° C., upon addition of instant composition, sample A, sample B, sample C, sample D, sample E and sample F, respectively.

FIG. 8 illustrates peroxide value of fish oil at 10° C., upon addition of instant composition and alpha-tocopherol, respectively.

FIG. 9 illustrates total polar compound of palm oil, upon addition of instant composition, sample A, sample B, sample C, sample D, sample E and sample F, respectively.

FIG. 10 illustrates total polar compound of sunflower oil, upon addition of instant composition, sample A, sample B, sample C, sample D, sample E and sample F, respectively.

FIG. 11 illustrates relationship between particle size and antioxidant activity

### DETAILED DESCRIPTION OF THE INVENTION

The present disclosure relates to a composition comprising green tea extract and rosemary extract, optionally along with adjuvant or excipient or a combination thereof.

In an embodiment the present disclosure relates to said green tea extract is at a concentration ranging from about 24% w/w to about 80% w/w.

In another embodiment the present disclosure relates to said rosemary extract is at a concentration ranging from about 45% w/w to about 85% w/w.

In yet another embodiment the present disclosure relates to said rosemary extract comprises rosemarinic acid at a concentration ranging from about 0.5% w/w to about 10% w/w, preferably at a range of about 0.5% w/w to about 3.5% w/w and carnosic acid at a concentration ranging from about 1.0% w/w to about 20% w/w, preferably at a range of about 1.5% w/w to about 12% w/w.

In still another embodiment the present disclosure relates to the adjuvant is selected from a group comprising vitamin C, gallic acid, vitamin E, rosmanol, ferulic acid, citric acid, mixed-tocopherol, lecithin, carotenoids and uric acid or any combination thereof.

In still another embodiment the present disclosure relates to the excipient is selected from a group comprising granulating agent, binding agent, lubricating agent, disintegrating agent, sweetening agent, glidant, anti-adherent, anti-static agent, surfactant, coating agent, colouring agent, flavouring agent, plasticizer, suspending agent, additive, emulsifying agent and spheronization agent or any combination thereof.

In still another embodiment the present disclosure relates to the emulsifying agent is polyglyceride fatty acid ester, preferably macrogoglycerol hydroxystearate; and wherein the additive is selected from a group comprising mono-di-glycerides, sorbitol, guar gum and xanthan gum or any combination thereof

In still another embodiment the present disclosure relates to said adjuvant at a concentration ranging from about 1.0 w/w to about 10% w/w, preferably ranging from about 1.5% w/w to about 8% w/w.

In still another embodiment the present disclosure relates to said excipient at a concentration ranging from about 0.5% to about 5% w/w, preferably ranging from about 1.0% w/w to about 2.5% w/w.

In still another embodiment the present disclosure relates to particle size of the composition ranging from about 5 μm to about 10 μm.

In still another embodiment the present disclosure relates to the composition optionally comprising polyphenols at a concentration ranging from about 30% to about 38%.

In still another embodiment the present disclosure relates to oil soluble composition having solubility ranging from about 95% to about 100% with settling less than about 0 about 5%.

The present disclosure further relates to a process of preparing the composition comprising green tea extract and rosemary extract, said process comprising acts of:
a. granulating crude extract of green tea and mixing the granulated extract with the rosemary extract to obtain a mixture;
b. optionally adding adjuvant or excipient or a combination thereof to the mixture; and
c. passing the mixture of step a) or b) through homogenizer to obtain said composition comprising green tea extract and rosemary extract

In an embodiment of the present disclosure, the crude extract of green tea having a particle size of about 50μ to about 200μ is granulated to obtain a extract of particle size ranging from about 10μ to about 20μ.

In another embodiment of the present disclosure, the composition obtained in step c) has a particle size ranging from about 5μ to about 10μ.

In yet another embodiment of the present disclosure, the crude extract of the green tea are granulated by techniques selected from a group comprising hammer mill, ball mill, plate mill, disc mill, colloid mill, micronisation method, high pressure homogenization and cryogenic grinding or a combination thereof, preferably cryogenic grinding.

In still another embodiment of the present disclosure, the mixing is carried out by an agitator at a speed of about 500 rpm to about 1000 rpm.

In still another embodiment of the present disclosure, the mixing is at a temperature ranging from about 40° C. to about 65° C., preferably at about 50° C. for time period ranging from about 2 hrs to about 24 hrs.

In still another embodiment of the present disclosure, the mixture is homogenized at a pressure ranging from about 500 bar to about 1000 bar.

The present disclosure further relates to a composition comprising the composition as mentioned above and tertiary butyl hydroquinone.

In an embodiment of the present disclosure, the composition as mentioned above is at a concentration ranging from about 50 ppm to about 100 ppm.

In another embodiment of the present disclosure, the tertiary butyl hydroquinone is at a concentration ranging from about 50 ppm to about 100 ppm.

The present disclosure further relates to an oil, fat or cosmetic preparation, comprising the composition of claim 1 or claim 20.

The present disclosure further relates to method of preparing the oil, fat or cosmetic preparation as claimed in claim 23, wherein said method comprises act of mixing the composition of claim 1 or claim 17 with the oil, fat or cosmetic ingredients.

The present invention provides an oil soluble composition comprising extract of green tea with rosemary extract, optionally along with adjuvants and excipients, wherein the composition is a natural antioxidant.

In an embodiment, the composition of the instant invention acts as a preservative in edible oil or cooking oil or food grade oil and oils which have cosmetic applications.

In an embodiment, the composition of the instant invention increases the oxidative induction time of the oil, thereby decreasing the rate of primary and secondary oxidation of the oil.

In an embodiment, the composition of the instant invention is added to oil at a concentration as low as about 50 ppm to as high as about 1000 ppm, unlike the conventionally known synthetic antioxidant, which have an upper limit of 200 ppm (under the regulatory guidelines) beyond which it is considered to be carcinogenic, whereas some antioxidant at higher dosage (above 500 ppm) acts as a pro-oxidant.

In an embodiment, the composition of the instant invention reduces the formation of polar compounds in the oil, thereby reducing degradation of oil. The composition of the instant invention also enhances the number of frying cycles at elevated temperatures and increases the shelf life of the oil.

In an embodiment, the oils for which the composition of instant invention acts as a preservative are avocado oil, mustard oil, palm oil, peanut oil, rice barn oil, safflower oil, sesame oil, sunflower oil, almond oil, canola oil, coconut oil, corn oil, cottonseed oil, mustard oil, grape seed oil, olive oil, pumpkin seed oil, tea seed oil, walnut oil, fish oil or any combination thereof.

In another embodiment, adjuvant of the instant composition is selected from a group comprising gallic acid, vitamin C, vitamin E, rosmanol, ferulic acid, citric acid, mixed-tocopherol, carotenoids and uric acid or any combination thereof. The excipient of the instant composition is selected from a group comprising granulating agent, binding agent, lubricating agent, disintegrating agent, sweetening agent, glidant, anti-adherent, anti-static agent, surfactant, coating agent, colouring agent, flavouring agent, plasticizer, suspending agent, emulsifying agent and spheronization agent or any combination thereof.

In an embodiment, the composition of the instant invention comprises about 24% w/w to about 80% w/w of green tea extract in powdered form and about 45% w/w to about 80% w/w of rosemary extract in oil form. The composition optionally comprises about 1% w/w to about 10% w/w, preferably from about 1.5% to about 8% w/w of one or more of adjuvants such as vitamin C, gallic acid, vitamin E, rosmanol, ferulic acid, citric acid, mixed-tocopherol, carotenoids, uric acid; optionally along with about 0.5% w/w to about 5% w/w, preferably from about 1.0% w/w to about 2.5% w/w excipients. The green tea extract in the composition of the instant invention is in the form of a powder made through cryogenic grinding with a particle size of about 10μ to about 200μ; and the rosemary is in the form of lipophilic extract containing rosemarinic acid at a concentration ranging from about 1.0% w/w to about 10% w/w, preferably from about 0.5% w/w to about 3.5% w/w and carnosic acid at a concentration ranging from about 1.0% w/w to about 20% w/w, preferably from about 1.5 w/w to about 12% w/w.

In an embodiment, the composition of the present invention comprises about 30% to about 38% of polyphenols, wherein the polyphenols content in the crude green tea extract is 90% and when this extract is cryo-grinded, mixed with rosemary extract optionally along with adjuvants and excipients, followed by high pressure homogenizing, the polyphenol content of PRESOL will be reduced to about 30% to about 38%, thereby enhancing the solubility of PRESOL.

In an embodiment, excipients, preferably, emulsifying agents enhances the solubility of the instant composition in oils, wherein the emulsifying agent is macrogoglycerol hydroxystearate. The instant composition comprising macrogogycerol hydroxystearate has a solubility ranging from about 95% to about 100% with settling less than about 5%.

In an embodiment, the instant composition comprises mono-di-glycerides as an additive.

In an embodiment, the composition of present invention is lipid soluble which prevents oxidative rancidity of oils and fats.

In an embodiment, the rosemary extract of the instant composition comprises rosemarinic acid or carnosic acid or a combination thereof.

The present invention further relates to a process of preparing a composition comprising extract of green tea with rosemary extract, optionally along with adjuvants and excipients, wherein said process comprises the following steps:
a) crude green tea extract with a particle size of 50μ to about 200μ is granulated to obtain a extract of particle size ranging from about 10μ to about 20μ;
b) an extract, sage or thyme of a Labiatae herb, preferably rosemary extract, is mixed with granulated green tea extract of particle size 10μ to about 20μ at a temperature ranging from about 40° C. to 65° C., preferably at about 50° C. for about 2 hrs to about 24 hrs, in a cylindrical vessel with an agitator rod with paddle that rotates at a speed of about 500 RPM to about 1200 RPM, to form a homogenous solution;
c) optionally adjuvants or excipients or a combination thereof is added to the solution, and mixed thoroughly to obtain a mixture;
d) the mixture of step d) is passed through high pressure homogenizer thrice at different pressures preferably in the range of about 500 bar to about 1000 bar, to obtain the final composition of the present invention.

In an embodiment, the green tea extract is granulated by any one or combination of the method selected from a group comprising hammer mill, ball mill, plate mill, disc mill, colloid mill, micronisation method, high pressure homogenization and cryogenic grinding.

In an embodiment, the crude green tea extract is granulated by cryogenic grinding to a particle size ranging from about 10μ to about 20μ

In an embodiment, the crude green tea extract before grinding is in the form of granular powder with a particle size ranging from about 50μ to about 200μ.

In an embodiment, the adjuvant is selected from a group comprising vitamin C, gallic acid, vitamin E, rosmanol, ferulic acid, citric acid, mixed-tocopherol, carotenoids and uric acid or any combination thereof.

In an embodiment, the excipient is selected from a group comprising granulating agent, binding agent, lubricating agent, disintegrating agent, sweetening agent, glidant, anti-adherent, anti-static agent, surfactant, coating agent, coloring agent, flavoring agent, plasticizer, suspending agent, emulsifying agent and spheronization agent or any combination thereof.

The present invention further relates to fortifying the composition comprising green tea extract and rosemary optionally along with adjuvant or excipients or a combination thereof with antioxidant selected from a group comprising butylated hydroxyanisole, butylated hydroxyl toluene, tertiary butyl hydroquinone, propyl gallate or any combination thereof, preferably butylated hydroxyl toluene.

In an embodiment, fortification of composition comprising green tea extract and rosemary extract optionally along with adjuvant or excipients or a combination thereof with antioxidant selected from a group comprising butylated hydroxyanisole, butylated hydroxyl toluene, tertiary butyl hydroquinone, propyl gallate or any combination thereof is carried out a concentration ranging from about 50 ppm to about 200 ppm, preferably in the range of about 50 ppm to about 100 ppm.

In an embodiment, present invention relates to an oil comprising green tea extract and rosemary extract, optionally along with adjuvant or excipients or a combination thereof, wherein the oil is edible oil or cooking oil or food grade oil, oil for cosmetic application, vegetable oil, plant oil or any combination thereof.

In another embodiment, present invention relates to an oil comprising green tea extract and rosemary extract, optionally along with adjuvant or excipients or a combination thereof, fortified with antioxidant selected from a group comprising butylated hydroxyanisole, butylated hydroxyl toluene, tertiary butyl hydroquinone, propyl gallate or any combination thereof, wherein the oil is edible oil or cooking oil or food grade oil, oil for cosmetic application, vegetable oil, plant oil or any combination thereof.

The further embodiment herein describes the scientific and technical terms used in connection with the instant invention and shall have meaning/definitions/equations/glossary that are commonly understood by those skilled in the art:

As used herein, ‘Oil Stability Index (OSI)’ is an American Oil Chemists Society (AOCS) approved method that determines the relative resistance of fat and oil samples to oxidation, which is defined by the following equation—

$$Oil stability index ( O S I ) = \frac{Induction time of treated oil}{Induction time of control oil}$$

As used herein, ‘increase in protection factor’ is defined by the following equation—

$$Increase in protection factor = \frac{O S I of treated oil - O S I of control oil}{O S I of control oil} \times 100$$

As used herein, ‘PRESOL’ is the final composition of the instant invention, comprising extract of green tea with rosemary extract, optionally along with adjuvants and excipients.

As used herein, ‘crude green tea extract’ is a commercially available extract, obtained from green tea leaves (Camellia sinensis) having 90% polyphenol.

As used herein, ‘rosemary extract’ is commercially available extract which comprises rosemarinic acid at a concentration ranging from about 0.5% w/w to about 10% w/w and carnosic acid at a concentration ranging from about 1.0% w/w to about 20% w/w.

As used herein, ‘control’ is an oil without any antioxidant.

As used herein, ‘Sample A’ is oil with TBHQ (98% activity)

As used herein, ‘Sample B’ is oil with BHA (98% activity)

As used herein, ‘Sample C’ is oil with pulverized green tea (95% polyphenols)

As used herein, ‘Sample D’ is oil with rosemary extract (8% Carnosic acid)

As used herein, ‘Sample E’ is oil with cryogrinded green tea (95% Polyphenols) As used herein, ‘Sample B1’ is oil with BHA (98% activity)

As used herein, ‘Sample B2’ is oil with alpha tocopherol (96% activity)

The present invention is further illustrated by the following examples. However, the following examples are provided for illustrative purposes only and are not intended to limit the scope of the invention.

### EXAMPLES

### Example 1

### Process of Preparing PRESOL

a. In order to prepare 1000 g of PRESOL, 500 g of crude green tea extract with a particle size of about 50μ to about 200μ is cryo-grinded for two cryo cycles for about 10 mins each with an intermediate cooling for about 1 minute. 470 g of green tea extract is recovered from the cryo-grinder, with a particle size of about 10μ to about 20μ. To 300 g of the cryo-grinded green tea extract, about 500 g of rosemary extract comprising 11.67% carnosic acid is added and mixed slowly in a cylindrical vessel with an agitator rod with paddle that rotates at a speed of about 500 RPM to about 1200 RPM at a temperature of about 40° C. to about 65° C. for about 2 hrs to about 24 hrs to obtain a mixture. To the mixture, 50 g of vitamin C, 50 g of Tocopherol, 50 g of lecithin, 90 g of mono-di-glycerides and 10 g of Macrogoglycerol hydroxystearate is added. This obtained mixture is mixed thoroughly and passed through a high pressure homogenizer at three different pressures in the range of about 500 bar to about 1000 bar to obtain PRESOL with a yield of about 93.5%.
b. In order to prepare 500 g of PRESOL, 250 g of crude green tea extract with a particle size of about 50μ to about 200μ is cryo-grinded for two cryo cycles for about 10 mins each with an intermediate cooling for about 1 minute. 235 g of green tea extract is recovered from the cryo-grinder, with a particle size of about 10μ to about 20μ. To 150 g of the cryo-grinded green tea extract, about 250 g of rosemary extract comprising 11.67% carnosic acid is added and mixed slowly in a cylindrical vessel with an agitator rod with paddle that rotates at a speed of about 500 RPM to about 1200 RPM at a temperature of about 40° C. to about 65° C. for about 2 hrs to about 24 hrs to obtain a mixture. This mixture is mixed thoroughly and passed through a high pressure homogenizer at three different pressures in the range of about 500 bar to about 1000 bar to obtain PRESOL with a yield of about 93.5%.

### Example 2

### Oil Stability Index

The oil stability index is determined using the Metrohm's Rancimat (Metrohm 743 Rancimat).

A. Oil Stability Index of Palm Oil

4.0 g of palm oil is weighed into the reaction vessel. The reaction vessel filled with oil is placed in a heating block. The temperature of the block is maintained at 100° C., 120° C. and 140° C., respectively. The oil is heated at the said temperatures, followed by passing air at a flow rate of 20 L/h. Flowing of air through the oil produces peroxides during primary oxidation and organic acids having low molecular weight, aldehydes and ketones with typical rancid odor during the secondary oxidation phase. These compounds are conveyed by the air flow into measurement vessel containing distilled water where conductivity is being checked continuously by sensor. Variation in conductivity of water shows the presence of organic acids, thus analyzing the induction time of the oil.

The above experiment is repeated by adding 200 ppm of TBHQ (sample-A), 200 ppm of BHA (sample-B), 200 ppm of pulverized green tea extract with 90% polyphenol (sample-C), 200 ppm of rosemary (sample-D), 200 ppm of green tea extract after cryogenic grinding (sample-E), 200 ppm of crude green tea extract (sample-F) and PRESOL at 200 ppm, 500 ppm and 1000 ppm, respectively to palm oil.

From the above experiment, oxidative stability index of untreated oil (control) and treated oil is calculated and the below table illustrates the induction time and the obtained oxidative stability.

TABLE 1
Oxidative Stability of Palm oil

| | | | | Oxidative Stability | | |
|---|---|---|---|---|---|---|
| | Induction Time (h) | | | Index (OSI) | | |
| Treatment | 100° C. | 120° C. | 140° C. | 100° C. | 120° C. | 140° C. |
| Control PO | 41.7 | 9.64 | 2.45 | 1.00 | 1.00 | 1.00 |
| Sample A | 95.76 | 20.93 | 5.26 | 2.30 | 2.17 | 2.15 |
| Sample B | 43.33 | 9.88 | 2.51 | 1.04 | 1.02 | 1.02 |
| Sample C | 74.22 | 17.95 | 4.48 | 1.78 | 1.86 | 1.83 |
| Sample D | 54.85 | 13.32 | 3.14 | 1.32 | 1.38 | 1.28 |
| Sample E | 82.46 | 20.02 | 4.95 | 1.98 | 2.08 | 2.02 |
| Sample F | 58.23 | 14.17 | 3.45 | 1.40 | 1.47 | 1.41 |
| PRESOL | 63.59 | 15.57 | 3.86 | 1.52 | 1.62 | 1.58 |
| PRESOL | 97.4 | 23.74 | 5.62 | 2.34 | 2.46 | 2.29 |
| PRESOL | 135.8 | 33.64 | 8.14 | 3.26 | 3.49 | 3.32 |

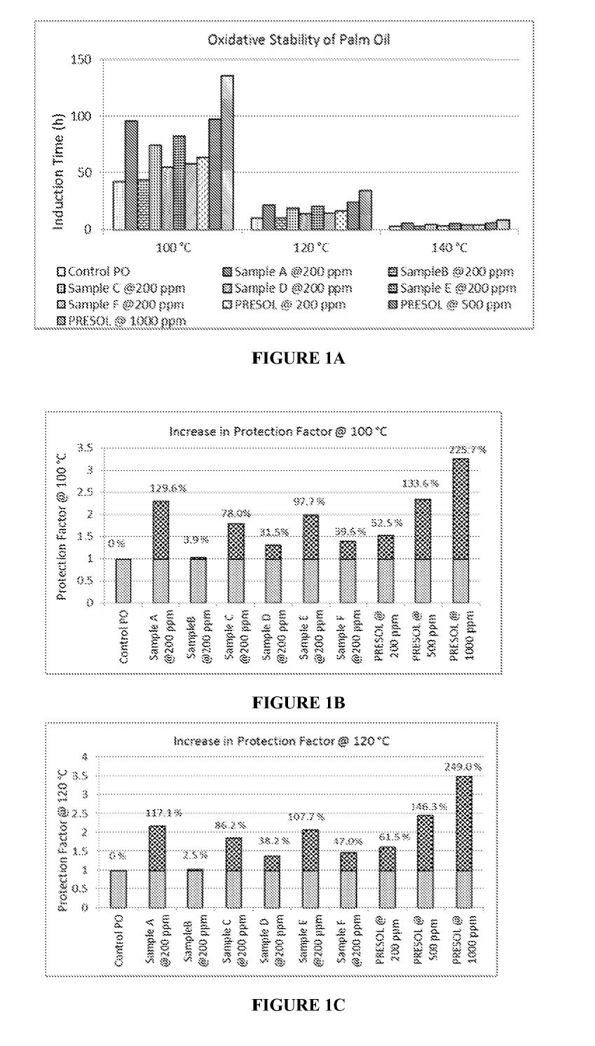

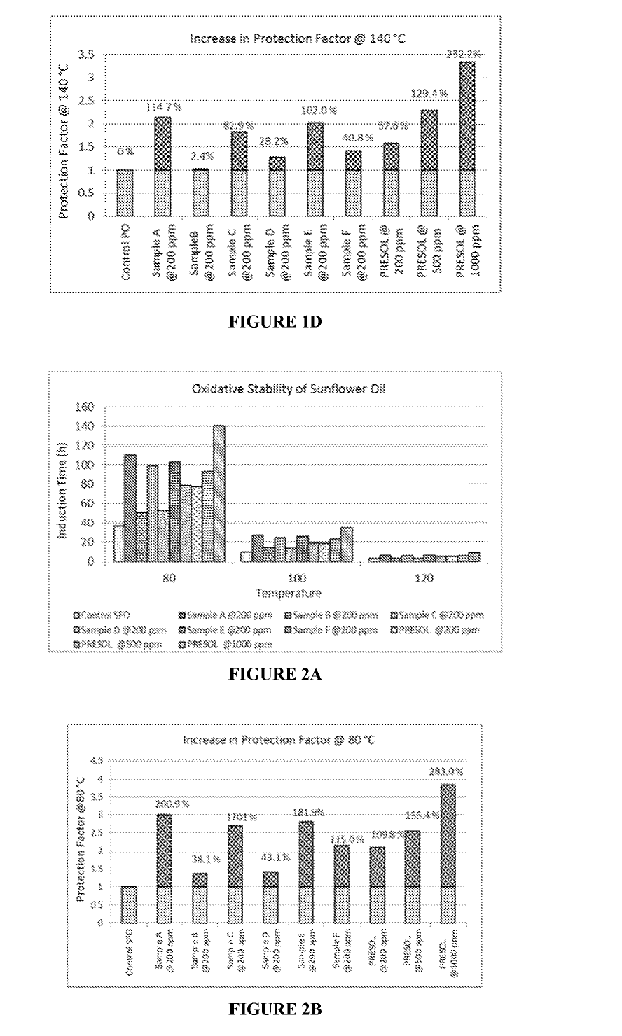

Based on the obtained oxidative stability index (illustrated in FIG. 1A), protection factor for the samples are assessed at temperatures 100° C., 120° C. and 140° C. to analyze the increase in the protection factor, which in turn illustrates the enhancement in oxidative stability obtained by treating the oil with PRESOL, TBHQ, BHA, pulverized green tea extract, rosemary extract, green tea extract after cryogenic grinding and crude green tea extract. FIGS. 1B, 1C and 1D illustrate the increase in the protection factor by treating the oil with PRESOL. Further table 1 illustrates substantial enhancement of oxidative stability of palm oil with PRESOL.

B. Oil Stability Index of Sunflower Oil

4.0 g of sunflower oil is weighed into the reaction vessel. The reaction vessel filled with oil is placed in a heating block. The temperature of the block is maintained at 80° C., 100° C. and 120° C., respectively. The oil is heated at the said temperatures, followed by passing air at a flow rate of 20 L/h. Flowing of air through the oil produces peroxides during primary oxidation and organic acids having low molecular weight, aldehydes and ketones with typical rancid odor during the secondary oxidation phase. These compounds are conveyed by the air flow into measurement vessel containing distilled water where conductivity is being checked continuously by sensor. Variation in conductivity of water shows the presence of organic acids, thus analyzing the induction time of the oil.

The above experiment is repeated by adding 200 ppm of TBHQ (sample-A), 200 ppm of BHA (sample-B), 200 ppm of pulverized green tea extract with 90% polyphenol (sample-C), 200 ppm of rosemary extract (sample-D), 200 ppm of green tea extract after cryogenic grinding (sample-E), 200 ppm of crude green tea extract (sample-F) and PRESOL at 200 ppm, 500 ppm and 1000 ppm, respectively to sunflower oil. From the above experiment, oxidative stability index of untreated oil (control) and treated oil is calculated and the below table illustrates the induction time and the obtained oxidative stability.

TABLE 2
Oxidative Stability index of sunflower oil

| | | | | Oxidative Stability | | |
|---|---|---|---|---|---|---|
| | Induction Time (h) | | | Index (OSI) | | |
| Treatment | 80° C. | 100° C. | 120° C. | 80° C. | 100° C. | 120° C. |
| Control SFO | 36.48 | 9.32 | 2.35 | 1.00 | 1.00 | 1.00 |
| Sample A | 109.76 | 26.82 | 6.13 | 3.01 | 2.88 | 2.61 |
| Sample B | 50.39 | 13.33 | 2.64 | 1.38 | 1.43 | 1.12 |
| Sample C | 98.52 | 24.03 | 5.48 | 2.70 | 2.58 | 2.33 |
| Sample D | 52.19 | 12.94 | 2.85 | 1.43 | 1.39 | 1.21 |
| Sample E | 102.85 | 25.28 | 6.05 | 2.82 | 2.71 | 2.57 |
| Sample F | 78.42 | 19.08 | 4.55 | 2.15 | 2.05 | 1.94 |
| PRESOL | 76.52 | 18.38 | 4.29 | 2.10 | 1.97 | 1.83 |
| PRESOL | 93.18 | 22.76 | 5.47 | 2.55 | 2.44 | 2.33 |
| PRESOL | 139.72 | 34.41 | 8.38 | 3.83 | 3.69 | 3.57 |

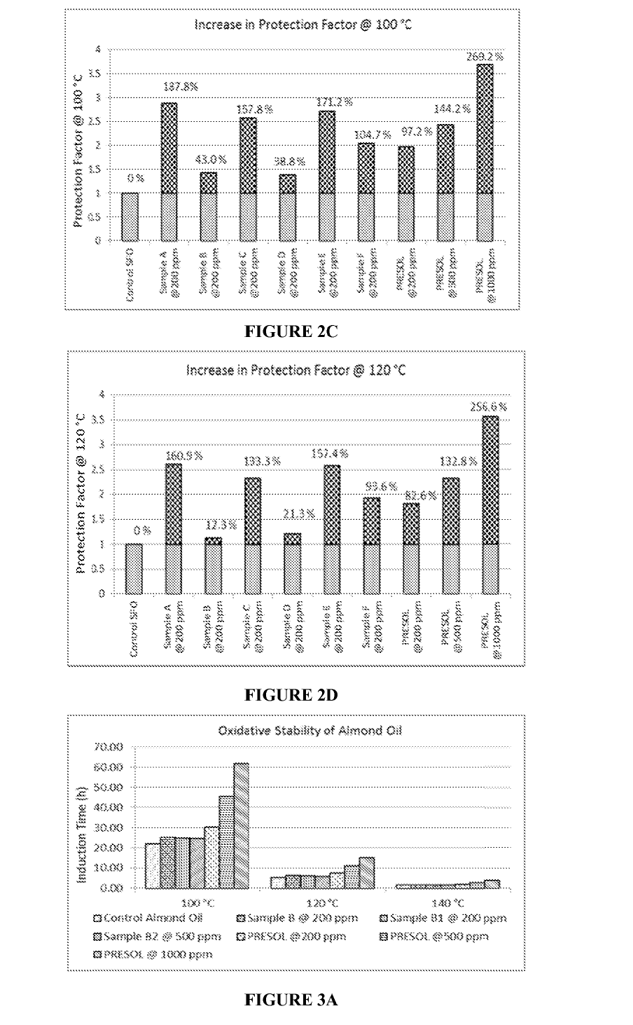

Based on the obtained oxidative stability index (illustrated in FIG. 2A), protection factor for the samples are assessed at temperatures 80° C., 100° C. and 120° C. to analyze the increase in the protection factor, which in turn illustrates the enhancement in oxidative stability obtained by treating the oil with PRESOL, TBHQ, BHA, pulverized green tea extract, rosemary extract, green tea extract after cryogenic grinding and crude green tea extract. FIGS. 2B, 2C and 2D illustrate the increase in the protection factor by treating sunflower oil with PRESOL. Further table 2 illustrates substantial enhancement of oxidative stability of palm oil with PRESOL.

Lower temperature is chosen to study the oxidative stability of sunflower oil, because sunflower is less stable at higher temperatures when compared to palm oil.

C. Oil Stability Index of Almond Oil

4.0 g of almond oil is weighed into the reaction vessel. The reaction vessel filled with oil is placed in a heating block. The temperature of the block is maintained at 100° C., 120° C. and 140° C., respectively. The oil is heated at the said temperatures, followed by passing air at a flow rate of 20 L/h. Flowing of air through the oil produces peroxides during primary oxidation and organic acids having low molecular weight, aldehydes and ketones with typical rancid odor during the secondary oxidation phase. These compounds are conveyed by the air flow into measurement vessel containing distilled water where conductivity is being checked continuously by sensor. Variation in conductivity of water shows the presence of organic acids, thus analyzing the induction time of the oil.

The above experiment is repeated by adding 200 ppm of BHA (sample-B), 200 ppm of BHT (sample-B1), 500 ppm of alpha tocopherol (sample-B2) and PRESOL at 200 ppm, 500 ppm and 1000 ppm, respectively to almond oil. From the above experiment, oxidative stability index of untreated oil (control) and treated oil is calculated and the below table illustrates the induction time and the obtained oxidative stability.

TABLE 3
Oxidative Stability index of almond oil

| | | | | Oxidative Stability | | |
|---|---|---|---|---|---|---|
| | Induction Time | | | Index (OSI) | | |
| Treatment | 100° C. | 120° C. | 140° C. | 100° C. | 120° C. | 140° C. |
| Control | 22.09 | 5.06 | 1.37 | 1.00 | 1.00 | 1.00 |
| Almond Oil | | | | | | |
| Sample B | 25.14 | 6.11 | 1.52 | 1.14 | 1.21 | 1.11 |
| Sample B1 | 24.98 | 5.89 | 1.48 | 1.13 | 1.16 | 1.08 |
| Sample B2 | 24.51 | 5.73 | 1.43 | 1.11 | 1.13 | 1.04 |
| PRESOL | 30.16 | 7.48 | 1.77 | 1.37 | 1.48 | 1.29 |
| PRESOL | 45.37 | 11.00 | 2.65 | 2.05 | 2.17 | 1.93 |
| PRESOL | 62.13 | 15.05 | 3.62 | 2.81 | 2.97 | 2.64 |

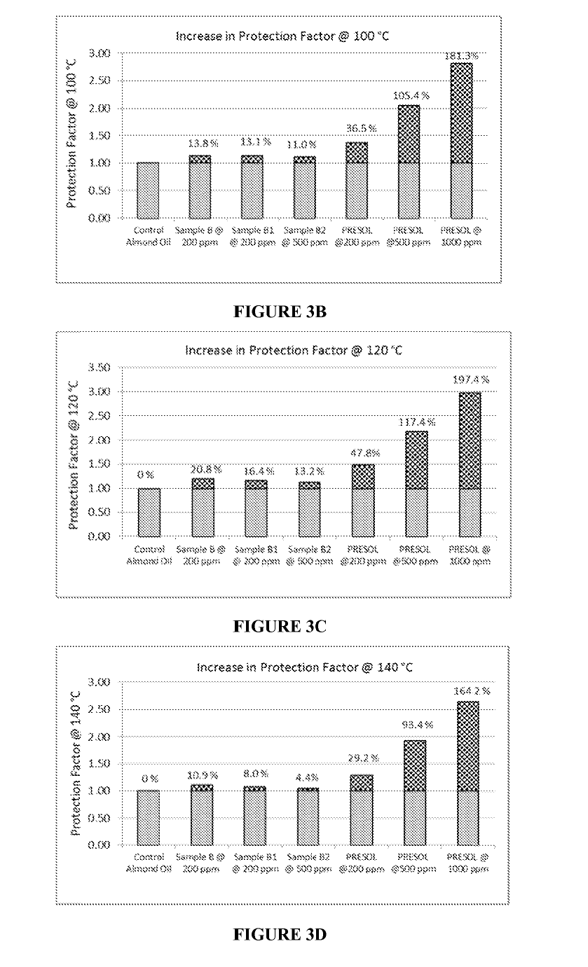

Based on the obtained oxidative stability index (illustrated in FIG. 3A), protection factor for the samples are assessed at temperatures 100° C., 120° C. and 140° C. to analyze the increase in the protection factor, which in turn illustrates the enhancement in oxidative stability obtained by treating the oil with PRESOL, BHA, BHT and alpha tocopherol. FIGS. 3B, 3C and 3D illustrate the increase in the protection factor by treating almond oil with PRESOL. Further table 3 illustrates substantial enhancement of oxidative stability of almond oil with PRESOL.

D. Oil Stability Index of Apricot Oil

4.0 g of apricot oil is weighed into the reaction vessel. The reaction vessel filled with oil is placed in a heating block. The temperature of the block is maintained at 100° C., 120° C. and 140° C., respectively. The oil is heated at the said temperatures, followed by passing air at a flow rate of 20 L/h. Flowing of air through the oil produces peroxides during primary oxidation and organic acids having low molecular weight, aldehydes and ketones with typical rancid odor during the secondary oxidation phase. These compounds are conveyed by the air flow into measurement vessel containing distilled water where conductivity is being checked continuously by sensor. Variation in conductivity of water shows the presence of organic acids, thus analyzing the induction time of the oil.

The above experiment is repeated by adding 200 ppm of BHA (sample-B), 200 ppm of BHT (sample-B1), 500 ppm of alpha tocopherol (sample-B2) and PRESOL at 200 ppm, 500 ppm and 1000 ppm, respectively to apricot oil. From the above experiment, oxidative stability index of untreated oil (control) and treated oil is calculated and the below table illustrates the induction time and the obtained oxidative stability.

TABLE 4
Oxidative Stability index of apricot oil

| | Induction Time | | | Oxidative Stability | | |
|---|---|---|---|---|---|---|
| | at 200 ppm | | | Index (OSI) | | |
| Treatment | 100° C. | 120° C. | 140° C. | 100° C. | 120° C. | 140° C. |
| Control | 26.88 | 5.87 | 1.48 | 1.00 | 1.00 | 1.00 |
| Apricot Oil | | | | | | |
| Sample B | 29.16 | 6.66 | 1.57 | 1.08 | 1.13 | 1.06 |
| Sample B1 | 26.96 | 5.93 | 1.49 | 1.00 | 1.01 | 1.01 |
| Sample B2 | 22.06 | 5.06 | 1.32 | 0.82 | 0.86 | 0.89 |
| PRESOL | 33.68 | 8.43 | 1.88 | 1.25 | 1.44 | 1.27 |
| PRESOL | 45.22 | 10.15 | 2.46 | 1.68 | 1.73 | 1.66 |
| PRESOL | 56.19 | 13.88 | 3.29 | 2.09 | 2.36 | 2.22 |

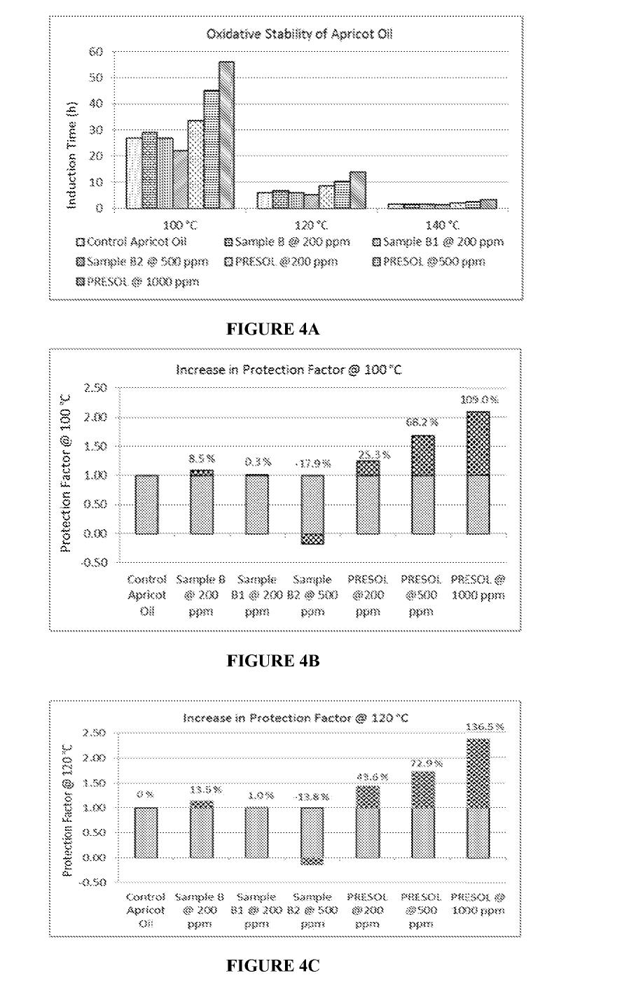

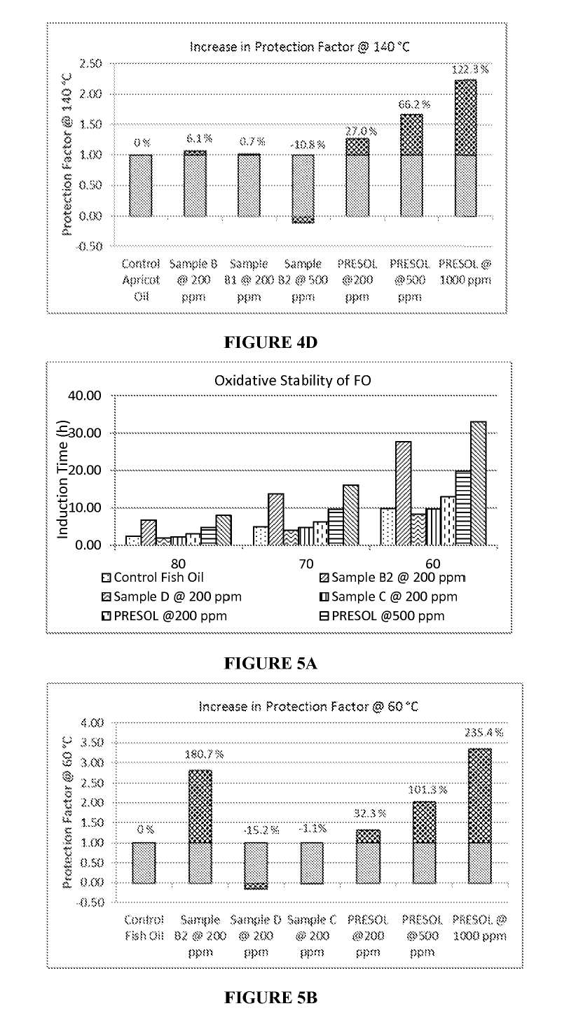

Based on the obtained oxidative stability index (illustrated in FIG. 4A), protection factor for the samples are assessed at temperatures 100° C., 120° C. and 140° C. to analyze the increase in the protection factor, which in turn illustrates the enhancement in oxidative stability obtained by treating the oil with PRESOL, BHA, BHT and alpha tocopherol. FIGS. 4B, 4C and 4D illustrate the increase in the protection factor by treating apricot oil with PRESOL. Further table 4 illustrates substantial enhancement of oxidative stability of apricot oil with PRESOL.

E. Oil Stability Index of Fish Oil

4.0 g of fish oil is weighed into the reaction vessel. The reaction vessel filled with oil is placed in a heating block. The temperature of the block is maintained at 60° C., 70° C. and 80° C., respectively. The oil is heated at the said temperatures, followed by passing air at a flow rate of 20 L/h. Flowing of air through the oil produces peroxides during primary oxidation and organic acids with low molecular weight, aldehydes and ketones with typical rancid odor during the secondary oxidation phase. These compounds are conveyed by the air flow into measurement vessel containing distilled water where conductivity is being checked continuously by sensor. Variation in conductivity of water shows the presence of organic acids, thus analyzing the induction time of the oil.

The above experiment is repeated by adding, 200 ppm of alpha tocopherol (sample-B2), 200 ppm of pulverized green tea extract with 90% polyphenol (sample-C), 200 ppm of rosemary extract (sample-D) and PRESOL at 200 ppm, 500 ppm and 1000 ppm, respectively to fish oil. From the above experiment, oxidative stability index of untreated oil (control) and treated oil is calculated and the below table illustrates the induction time and the obtained oxidative stability.

TABLE 5
Oxidative Stability index of Fish oil

| | | | | Oxidative Stability | | |
|---|---|---|---|---|---|---|
| | Induction Time | | | Index (OSI) | | |
| Treatment | 80° C. | 70° C. | 60° C. | 80° C. | 70° C. | 60° C. |
| Control Fish oil | 2.46 | 4.98 | 9.86 | 1.00 | 1.00 | 1.00 |
| Sample B2 | 6.78 | 13.74 | 27.68 | 2.76 | 2.76 | 2.81 |
| Sample D | 1.97 | 4.05 | 8.36 | 0.80 | 0.81 | 0.85 |
| Sample C | 2.28 | 4.74 | 9.75 | 0.93 | 0.95 | 0.99 |
| PRESOL at | 3.07 | 6.32 | 13.04 | 1.25 | 1.27 | 1.32 |
| 200 ppm | | | | | | |
| PRESOL at | 4.78 | 9.68 | 19.85 | 1.94 | 1.94 | 2.01 |
| 500 ppm | | | | | | |
| PRESOL at | 8.02 | 16.14 | 33.07 | 3.26 | 3.24 | 3.35 |
| 1000 ppm | | | | | | |

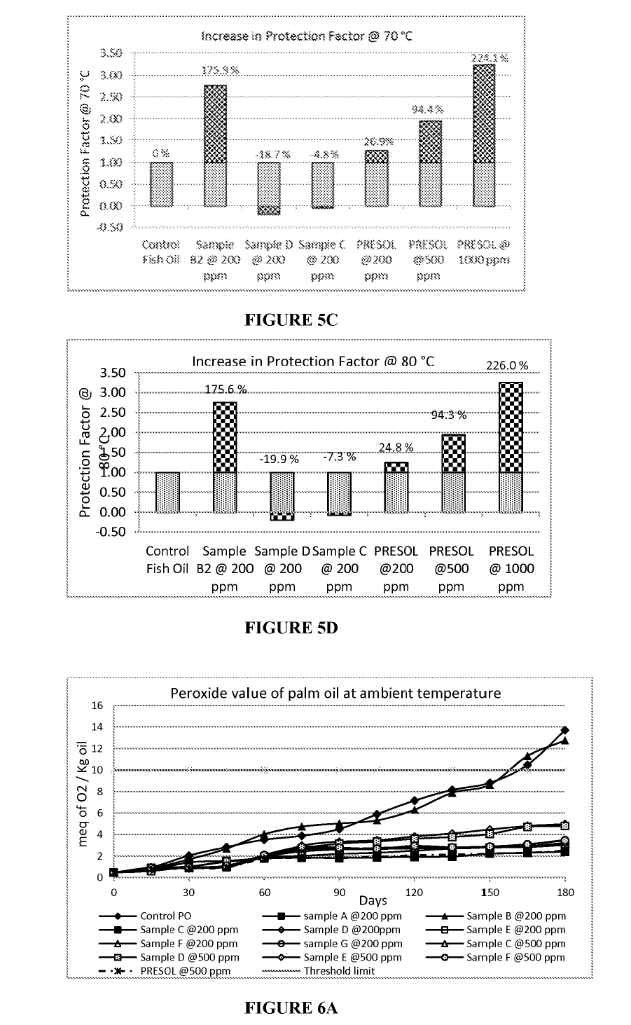

Based on the obtained oxidative stability index (illustrated in FIG. 5A), protection factor for the samples are assessed at temperatures 60° C., 70° C. and 80° C. to analyze the increase in the protection factor, which in turn illustrates the enhancement in oxidative stability obtained by treating the oil with PRESOL, alpha tocopherol, pulverized green tea extract with 90% polyphenol and rosemary extract. FIGS. 5B, 5C and 5D illustrate the increase in the protection factor by treating fish oil with PRESOL. Further table 5 illustrates substantial enhancement of oxidative stability of fish oil with PRESOL.

### Example 3

An extension of the Example 1 is provided by a new function known as temperature extrapolation. This is an aid for estimating the shelf life of oils and fats. The extrapolation makes use of the relationship between the measured induction time and the temperature given by van't Hoff's law. Several measurements are made at different temperatures and then extrapolated to the storage temperature. The values so obtained allow estimation of the storage stability of the oil or fat containing PRESOL.

The shelf life is calculated using Rancimat shelf life calculator. The shelf life of oil is studied by carrying out chemical analysis, which include studying peroxide value (represents extent of primary oxidation), Para-anisidine value (represents extent of secondary oxidation) and totox value (reflects total oxidation of oil). These parameters are determined for 1 kg of untreated oil and treated oil at every 15 days interval for samples stored at ambient temperature and at every 7 days interval for samples stored at 50° C. The treated oil being, the oil upon addition of 200 ppm of TBHQ (sample-A), 200 ppm of BHA (sample-B), 200 ppm and 500 ppm of pulverized green tea extract with 90% polyphenol (sample-C), 200 ppm and 500 ppm of rosemary extract (sample-D), 200 ppm and 500 ppm of green tea extract after cryogenic grinding (sample-E), 200 ppm and 500 ppm of crude green tea extract (sample-F) and PRESOL at 200 ppm and 500 ppm. The untreated oil is the control sample without any antioxidant.

The threshold limit for per oxide value under which oil can be used for safe consumption is 10 meq of O2/Kg oil. Similarly, the threshold limit of para-anisidine value and Totox value is 10 and 30, respectively.

A. Determination of Shelf Life of Palm Oil

TABLE 6
Estimation of Shelf life of Palm oil

| Treatment | Shelf Life (days) at 30° C. |
|---|---|
| Control | 242.9 |
| Sample A | 617.0 |
| Sample B | 260.9 |
| Sample C | 420.8 |
| Sample D | 337.8 |
| Sample E | 475.1 |
| Sample F | 337.4 |
| PRESOL | 356.7 |
| PRESOL | 606.0 |
| PRESOL | 790.8 |

Table 6 illustrates that PRESOL suitably enhances the shelf life of palm oil.

TABLE 7
Peroxide value of palm oil at ambient temperature

| | | sample | Sample | Sample | | | | | | | | | |
|---|---|---|---|---|---|---|---|---|---|---|---|---|---|
| | | A | B | C | Sample D | Sample E | Sample | PRESOL | Sample C | Sample D | Sample E | Sample F | PRESOL |
| | Control | at 200 | at 200 | at 200 | at 200 | at 200 | F at 200 | at 200 | at 500 | at 500 | at 500 | at 500 | at 500 |
| Day | PO | ppm | ppm | ppm | ppm | ppm | ppm | ppm | ppm | ppm | ppm | ppm | ppm |
| 0 | 0.465 | 0.465 | 0.465 | 0.465 | 0.465 | 0.465 | 0.465 | 0.465 | 0.465 | 0.465 | 0.465 | 0.465 | 0.465 |
| 15 | 0.948 | 0.762 | 0.868 | 0.904 | 0.903 | 0.92 | 0.941 | 0.817 | 0.842 | 0.61 | 0.87 | 0.903 | 0.77 |
| 30 | 2.053 | 1.417 | 1.691 | 0.955 | 1.023 | 0.94 | 0.968 | 0.871 | 0.907 | 1.018 | 0.969 | 0.974 | 0.85 |
| 45 | 2.838 | 1.558 | 2.684 | 1.015 | 1.478 | 0.973 | 1.104 | 0.995 | 1.037 | 1.488 | 0.995 | 1.098 | 0.949 |
| 60 | 3.523 | 1.771 | 4.034 | 1.956 | 1.834 | 1.912 | 2.074 | 1.864 | 1.946 | 1.893 | 2.016 | 2.101 | 1.753 |
| 75 | 3.879 | 1.855 | 4.748 | 2.626 | 2.973 | 2.62 | 2.793 | 2.008 | 2.425 | 2.714 | 2.725 | 2.86 | 1.823 |
| 90 | 4.51 | 1.814 | 5.034 | 2.635 | 3.336 | 2.621 | 2.673 | 2.205 | 2.622 | 3.154 | 2.664 | 2.787 | 1.856 |
| 105 | 5.893 | 1.88 | 5.316 | 2.68 | 3.463 | 2.671 | 2.67 | 2.305 | 2.646 | 3.347 | 2.754 | 2.68 | 1.934 |
| 120 | 7.163 | 1.91 | 6.307 | 2.735 | 3.826 | 2.715 | 2.763 | 2.5 | 2.766 | 3.613 | 2.866 | 2.957 | 2.076 |
| 135 | 8.15 | 1.951 | 7.887 | 2.764 | 4.096 | 2.75 | 2.807 | 2.701 | 2.702 | 3.776 | 2.803 | 2.811 | 2.143 |
| 150 | 8.807 | 2.222 | 8.617 | 2.798 | 4.471 | 2.788 | 2.901 | 2.802 | 2.812 | 4.051 | 2.825 | 2.866 | 2.224 |
| 165 | 10.481 | 2.31 | 11.307 | 2.886 | 4.804 | 2.879 | 3.092 | 2.832 | 2.917 | 4.717 | 2.942 | 3.094 | 2.309 |
| 180 | 13.712 | 2.403 | 12.75 | 3.229 | 4.967 | 3.149 | 3.505 | 3.032 | 3.127 | 4.787 | 3.218 | 3.457 | 2.501 |

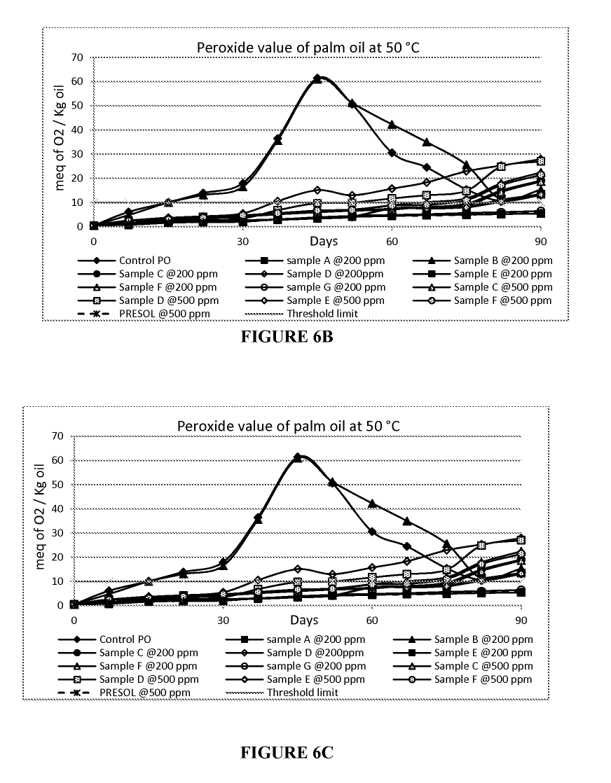

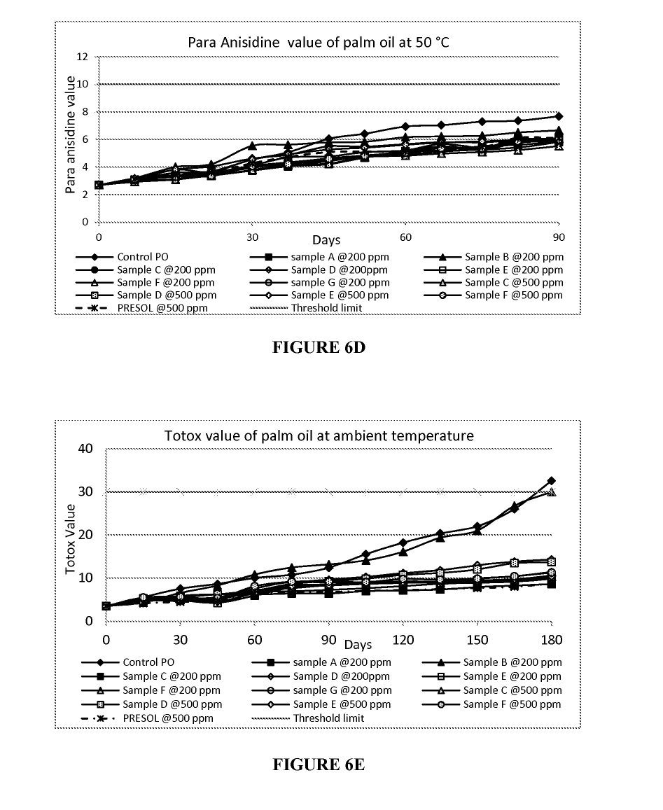

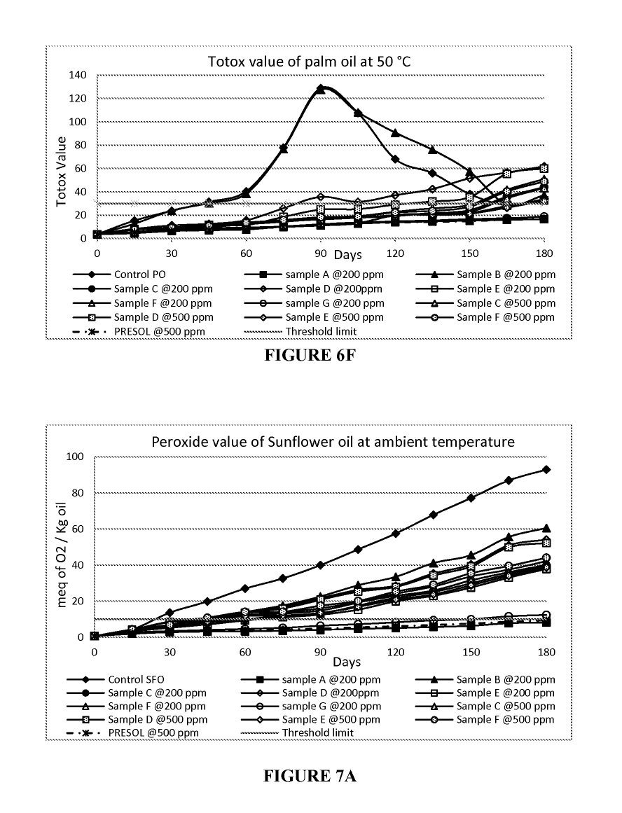

Table 7 and FIG. 6A illustrates that PRESOL at 500 ppm is showing equivalent efficacy in preventing oxidative rancidity as compared to 200 ppm of sample A in palm oil at ambient temperatures. From tables 6 and 7, it is further evident that 500 ppm PRESOL is having efficacy equivalent to 200 ppm Sample A. On the other hand control and sample B are crossing threshold limit after 165 days.

TABLE 8
Peroxide value of palm oil at 50° C.

| | | sample | Sample | Sample | | | | | | | | | |
|---|---|---|---|---|---|---|---|---|---|---|---|---|---|
| | | A | B | C | Sample D | Sample | Sample | PRESOL | Sample | Sample | Sample | Sample | PRESOL |
| | Control | at 200 | at 200 | at 200 | at | E at | F at | at 200 | C at | D at | E at | F at 500 | at 500 |
| Day | PO | ppm | ppm | ppm | 200 ppm | 200 ppm | 200 ppm | ppm | 500 ppm | 500 ppm | 500 ppm | ppm | ppm |
| 0 | 0.482 | 0.482 | 0.482 | 0.482 | 0.482 | 0.482 | 0.482 | 0.482 | 0.482 | 0.482 | 0.482 | 0.482 | 0.482 |
| 7 | 6.11 | 1.309 | 4.735 | 1.355 | 2.52 | 0.743 | 2.266 | 1.272 | 1.358 | 2.516 | 0.752 | 2.249 | 1.216 |
| 15 | 10.076 | 1.836 | 9.973 | 2.558 | 3.711 | 1.63 | 3.019 | 2.053 | 2.606 | 2.483 | 1.585 | 2.867 | 1.705 |
| 22 | 13.901 | 1.887 | 13.062 | 3.155 | 4.26 | 2.077 | 4.044 | 2.657 | 3.046 | 4.084 | 2 | 3.657 | 1.906 |
| 30 | 17.929 | 2.005 | 16.45 | 4.166 | 5.485 | 2.601 | 4.677 | 2.693 | 4.344 | 3.508 | 2.584 | 4.686 | 2.123 |
| 37 | 36.394 | 3.074 | 35.595 | 5.016 | 10.528 | 2.892 | 5.606 | 3.016 | 5.115 | 6.806 | 2.806 | 5.563 | 2.966 |
| 45 | 61.424 | 3.686 | 60.884 | 6.11 | 15.1 | 3.431 | 6.693 | 3.903 | 5.944 | 9.745 | 3.387 | 6.635 | 3.503 |
| 52 | 50.607 | 4.102 | 51.15 | 6.562 | 12.969 | 4.201 | 7.062 | 4.485 | 6.589 | 9.892 | 4.027 | 6.868 | 4.106 |
| 60 | 30.568 | 4.491 | 42.269 | 7.485 | 15.711 | 7.583 | 8.848 | 4.891 | 7.053 | 11.688 | 7.482 | 8.656 | 4.505 |
| 67 | 24.515 | 4.75 | 34.974 | 8.064 | 18.284 | 7.884 | 10.141 | 5.159 | 8.238 | 13.004 | 7.49 | 9.022 | 4.639 |
| 75 | 15.389 | 4.893 | 25.576 | 9.503 | 22.92 | 8.277 | 11.689 | 5.714 | 8.726 | 14.769 | 7.945 | 10.885 | 4.911 |
| 82 | 10.65 | 5.209 | 11.31 | 14.953 | 25.251 | 11.106 | 17.905 | 5.955 | 14.342 | 24.801 | 10.292 | 17.121 | 5.484 |
| 90 | 12.991 | 5.395 | 15.231 | 19.053 | 27.907 | 13.438 | 22.441 | 6.502 | 18.637 | 26.983 | 13.188 | 21.396 | 5.813 |

Table 8 and FIG. 6B illustrates that after 90 days of storage at 50° C., PRESOL at 200 ppm and 500 ppm does not cross threshold limit and are acting similar to sample A.

TABLE 9
Para-anisidine value of palm oil at ambient temperature

| | | sample | Sample | Sample | Sample | Sample | Sample | PRESOL | Sample | Sample | Sample | Sample | PRESOL |
|---|---|---|---|---|---|---|---|---|---|---|---|---|---|
| | | A at | B at | C at | D at | E at | F at | at | C at | D at | E at | F at | at |
| | Control | 200 | 200 | 200 | 200 | 200 | 200 | 200 | 500 | 500 | 500 | 500 | 500 |
| Day | PO | ppm | ppm | ppm | ppm | ppm | ppm | ppm | ppm | ppm | ppm | ppm | ppm |
| 0 | 2.566 | 2.566 | 2.566 | 2.566 | 2.566 | 2.566 | 2.566 | 2.566 | 2.566 | 2.566 | 2.566 | 2.566 | 2.566 |
| 15 | 3.656 | 3.062 | 2.602 | 3.472 | 3.458 | 3.221 | 3.563 | 3.032 | 3.507 | 3.453 | 3.25 | 3.664 | 2.607 |
| 30 | 3.435 | 3.045 | 3.18 | 3.265 | 2.569 | 2.966 | 3.403 | 3.077 | 3.343 | 3.497 | 3.367 | 3.681 | 2.772 |
| 45 | 2.957 | 3.113 | 2.899 | 3.178 | 2.603 | 2.302 | 3.309 | 3.096 | 3.465 | 3.259 | 2.562 | 3.111 | 2.851 |
| 60 | 2.973 | 2.615 | 2.759 | 3.008 | 2.74 | 2.111 | 3.434 | 3.003 | 3.302 | 3.256 | 2.459 | 3.89 | 2.87 |
| 75 | 3.017 | 2.731 | 2.952 | 3.053 | 2.86 | 2.613 | 3.525 | 2.923 | 2.809 | 3.218 | 2.662 | 3.439 | 3.033 |
| 90 | 3.383 | 2.772 | 3.113 | 3.157 | 2.946 | 3.038 | 3.561 | 2.831 | 3.089 | 2.958 | 3.024 | 3.621 | 3.027 |
| 105 | 3.752 | 3.217 | 3.455 | 3.389 | 3.347 | 3.315 | 3.683 | 3.022 | 3.324 | 3.157 | 3.184 | 3.701 | 3.158 |
| 120 | 3.906 | 3.303 | 3.499 | 3.548 | 3.502 | 3.427 | 3.72 | 3.136 | 3.472 | 3.526 | 3.338 | 3.873 | 3.073 |
| 135 | 4.046 | 3.438 | 3.593 | 3.654 | 3.626 | 3.614 | 3.628 | 3.256 | 3.484 | 3.617 | 3.493 | 4.006 | 3.136 |
| 150 | 4.389 | 3.473 | 3.759 | 3.869 | 4.049 | 3.711 | 4.15 | 3.33 | 3.799 | 3.896 | 3.697 | 4.106 | 3.236 |
| 165 | 5.05 | 3.639 | 4.169 | 3.934 | 4.231 | 3.781 | 4.228 | 3.468 | 3.87 | 4.014 | 3.835 | 4.213 | 3.367 |
| 180 | 5.144 | 3.808 | 4.493 | 4.109 | 4.363 | 3.891 | 4.394 | 3.764 | 4.08 | 4.127 | 3.944 | 4.364 | 3.665 |

TABLE 10
Para-anisidine value of palm oil at 50° C.

| | | sample | Sample | Sample | Sample | Sample | Sample | PRESOL | Sample | Sample | Sample | Sample | PRESOL |
|---|---|---|---|---|---|---|---|---|---|---|---|---|---|
| | | A at | B at | C at | D at | E at | F at | at | C at | D at | E at | F at | at |
| | Control | 200 | 200 | 200 | 200 | 200 | 200 | 200 | 500 | 500 | 500 | 500 | 500 |
| Day | PO | ppm | ppm | ppm | ppm | ppm | ppm | ppm | ppm | ppm | ppm | ppm | ppm |
| 0 | 2.69 | 2.69 | 2.69 | 2.69 | 2.69 | 2.69 | 2.69 | 2.69 | 2.69 | 2.69 | 2.69 | 2.69 | 2.69 |
| 7 | 3.143 | 3.062 | 3.186 | 3.006 | 3.163 | 3.041 | 3.169 | 2.975 | 2.932 | 3.048 | 3.092 | 3.104 | 3.117 |
| 15 | 3.585 | 3.296 | 4.011 | 3.337 | 3.819 | 3.293 | 3.417 | 3.124 | 3.09 | 3.329 | 3.814 | 3.301 | 3.437 |
| 22 | 3.686 | 3.395 | 4.204 | 3.449 | 4.042 | 3.343 | 3.595 | 3.489 | 3.397 | 3.462 | 3.641 | 3.369 | 3.608 |
| 30 | 4.545 | 3.74 | 5.542 | 4.033 | 4.62 | 4.036 | 4.121 | 3.95 | 3.782 | 4.066 | 4.546 | 4.089 | 4.273 |
| 37 | 5.062 | 4.052 | 5.617 | 4.303 | 4.886 | 4.104 | 4.694 | 4.266 | 4.144 | 4.337 | 4.911 | 4.208 | 4.727 |
| 45 | 6.054 | 4.209 | 5.794 | 4.636 | 5.502 | 4.433 | 4.771 | 4.455 | 4.249 | 4.606 | 5.271 | 4.589 | 5.085 |
| 52 | 6.41 | 4.685 | 5.827 | 4.835 | 5.475 | 4.788 | 5.075 | 4.806 | 4.716 | 4.807 | 5.397 | 4.809 | 5.121 |
| 60 | 6.942 | 5.091 | 6.165 | 5.079 | 5.681 | 4.96 | 5.205 | 4.92 | 4.822 | 5.137 | 5.614 | 5.052 | 5.155 |
| 67 | 7.035 | 5.208 | 6.22 | 5.423 | 5.798 | 5.385 | 5.674 | 5.12 | 4.975 | 5.501 | 5.775 | 5.365 | 5.639 |
| 75 | 7.29 | 5.514 | 6.269 | 5.271 | 5.789 | 5.233 | 5.417 | 5.271 | 5.089 | 5.3 | 5.862 | 5.35 | 5.454 |
| 82 | 7.36 | 5.601 | 6.503 | 5.942 | 5.98 | 5.773 | 5.996 | 5.411 | 5.225 | 5.877 | 5.949 | 5.88 | 6.118 |
| 90 | 7.676 | 5.827 | 6.657 | 5.956 | 6.092 | 5.866 | 6.048 | 5.818 | 5.521 | 6.047 | 6.074 | 5.993 | 6.096 |

Tables 9 and 10 illustrates the para-anisidine value of palm oil with PRESOL, TBHQ, BHA, pulverized green tea extract, crude green tea extract, cryogrinded green tea extract, rosemary extract. From the tables it can be observed that the para-anisidine values are within the threshold limits (10 meq of O₂/Kg oil). This is because, para-anisidine values reflects secondary oxidation of oil and since primary oxidation would still be under process, hence secondary oxidation products would not be formed and thereby the para-anisidine values are within the limit.

TABLE 11
Totox value of palm oil at ambient temperature

| | | sample | Sample | Sample | Sample | Sample | Sample | PRESOL | Sample | Sample | Sample | Sample | PRESOL |
|---|---|---|---|---|---|---|---|---|---|---|---|---|---|
| | | A at | B at | C at | D at | E at | F at | at | C at | D at | E at | F at | at |
| | Control | 200 | 200 | 200 | 200 | 200 | 200 | 200 | 500 | 500 | 500 | 500 | 500 |
| Day | PO | ppm | ppm | ppm | ppm | ppm | ppm | ppm | ppm | ppm | ppm | ppm | ppm |
| 0 | 3.497 | 3.497 | 3.497 | 3.497 | 3.497 | 3.497 | 3.497 | 3.497 | 3.497 | 3.497 | 3.497 | 3.497 | 3.497 |
| 15 | 5.551 | 4.585 | 4.338 | 5.280 | 5.265 | 5.060 | 5.445 | 4.666 | 5.190 | 4.673 | 4.991 | 5.470 | 4.146 |
| 30 | 7.540 | 5.879 | 6.561 | 5.175 | 4.615 | 4.845 | 5.338 | 4.818 | 5.157 | 5.534 | 5.305 | 5.628 | 4.471 |
| 45 | 8.634 | 6.228 | 8.268 | 5.208 | 5.560 | 4.248 | 5.517 | 5.086 | 5.538 | 6.234 | 4.552 | 5.307 | 4.748 |
| 60 | 10.019 | 6.157 | 10.827 | 6.919 | 6.408 | 5.934 | 7.582 | 6.731 | 7.194 | 7.042 | 6.490 | 8.092 | 6.376 |
| 75 | 10.775 | 6.441 | 12.449 | 8.306 | 8.807 | 7.854 | 9.110 | 6.939 | 7.659 | 8.647 | 8.112 | 9.159 | 6.678 |
| 90 | 12.403 | 6.400 | 13.181 | 8.427 | 9.617 | 8.279 | 8.907 | 7.241 | 8.332 | 9.267 | 8.352 | 9.195 | 6.738 |
| 105 | 15.539 | 6.976 | 14.088 | 8.749 | 10.273 | 8.656 | 9.023 | 7.631 | 8.615 | 9.851 | 8.692 | 9.062 | 7.026 |
| 120 | 18.232 | 7.122 | 16.114 | 9.018 | 11.154 | 8.857 | 9.245 | 8.137 | 9.004 | 10.753 | 9.070 | 9.787 | 7.225 |
| 135 | 20.347 | 7.339 | 19.368 | 9.183 | 11.817 | 9.113 | 9.241 | 8.658 | 8.889 | 11.169 | 9.099 | 9.629 | 7.422 |
| 150 | 22.002 | 7.916 | 20.994 | 9.466 | 12.991 | 9.286 | 9.953 | 8.934 | 9.424 | 11.998 | 9.347 | 9.839 | 7.683 |
| 165 | 26.012 | 8.258 | 26.783 | 9.707 | 13.839 | 9.539 | 10.411 | 9.133 | 9.705 | 13.448 | 9.718 | 10.402 | 7.984 |
| 180 | 32.567 | 8.614 | 29.993 | 10.567 | 14.297 | 10.188 | 11.404 | 9.828 | 10.335 | 13.701 | 10.380 | 11.278 | 8.666 |

Table 11 and FIG. 6E illustrates that control and sample B has crossed the threshold limit after 180 days as in case of peroxide value. On the other hand PRESOL at 500 ppm and Sample A at 200 ppm are performing equally.

TABLE 12
Totox value of palm oil at 50° C.

| | | sample | Sample | Sample | Sample | Sample | Sample | PRESOL | Sample | Sample | Sample | Sample | PRESOL |
|---|---|---|---|---|---|---|---|---|---|---|---|---|---|
| | | A at | B at | C at | D at | E at | F at | at | C at | D at | E at | F at | at |
| | Control | 200 | 200 | 200 | 200 | 200 | 200 | 200 | 500 | 500 | 500 | 500 | 500 |
| Day | PO | ppm | ppm | ppm | ppm | ppm | ppm | ppm | ppm | ppm | ppm | ppm | ppm |
| 0 | 3.655 | 3.655 | 3.655 | 3.655 | 3.655 | 3.655 | 3.655 | 3.655 | 3.655 | 3.655 | 3.655 | 3.655 | 3.655 |
| 15 | 15.364 | 5.679 | 12.656 | 5.716 | 8.203 | 4.528 | 7.701 | 5.520 | 5.764 | 8.124 | 4.608 | 7.615 | 5.364 |
| 30 | 23.737 | 6.967 | 23.957 | 8.454 | 11.242 | 6.553 | 9.454 | 7.230 | 8.542 | 8.780 | 6.472 | 9.170 | 6.500 |
| 45 | 31.488 | 7.169 | 30.328 | 9.759 | 12.562 | 7.498 | 11.683 | 8.803 | 9.553 | 11.809 | 7.369 | 10.922 | 7.210 |
| 60 | 40.404 | 7.750 | 38.442 | 12.365 | 15.590 | 9.239 | 13.475 | 9.336 | 12.755 | 11.563 | 9.257 | 13.644 | 8.028 |
| 75 | 77.850 | 10.200 | 76.806 | 14.336 | 25.942 | 9.889 | 15.906 | 10.298 | 14.567 | 18.523 | 9.820 | 15.852 | 10.077 |
| 90 | 128.903 | 11.581 | 127.563 | 16.856 | 35.701 | 11.295 | 18.157 | 12.261 | 16.494 | 24.761 | 11.362 | 18.354 | 11.256 |
| 105 | 107.624 | 12.888 | 108.126 | 17.959 | 31.412 | 13.190 | 19.199 | 13.775 | 17.984 | 25.182 | 12.863 | 18.857 | 12.929 |
| 120 | 68.077 | 14.074 | 90.702 | 20.048 | 37.103 | 20.125 | 22.902 | 14.702 | 19.243 | 28.990 | 20.016 | 22.468 | 13.833 |
| 135 | 56.064 | 14.709 | 76.168 | 21.551 | 42.365 | 21.152 | 25.957 | 15.439 | 21.976 | 31.783 | 20.345 | 23.683 | 14.255 |
| 150 | 38.068 | 15.300 | 57.420 | 24.276 | 51.629 | 21.786 | 28.796 | 16.699 | 22.753 | 35.399 | 21.239 | 27.223 | 14.912 |
| 165 | 28.660 | 16.019 | 29.123 | 35.849 | 56.482 | 27.985 | 41.807 | 17.322 | 34.561 | 55.551 | 26.465 | 40.359 | 16.193 |
| 180 | 33.658 | 16.617 | 37.118 | 44.062 | 61.906 | 32.742 | 50.931 | 18.821 | 43.321 | 60.041 | 32.369 | 48.888 | 17.146 |

Table 12 and FIG. 6F illustrates that control and sample B has crossed the threshold limit after 180 days as in case of peroxide value. On the other hand PRESOL at 500 ppm and Sample A at 200 ppm are performing equally.

B. Determination of Shelf Life of Sunflower Oil

TABLE 13
Estimation of Shelf life of sunflower oil

| Treatment | Shelf Life (days) at 30° C. |
|---|---|
| Control SFO | 46.3 |
| Sample A at 200 ppm | 171.0 |
| Sample B at 200 ppm | 87.2 |
| Sample C at 200 ppm | 137.6 |
| Sample Dat 200 ppm | 83.2 |
| Sample Eat 200 ppm | 147.8 |
| Sample Fat 200 ppm | 115.7 |
| PRESOL at 200 ppm | 117.5 |
| PRESOL at 500 ppm | 134.2 |
| PRESOL at 1000 ppm | 198.5 |

TABLE 14
Peroxide value of sunflower oil at ambient temperatures

| | | sample | Sample | Sample | Sample | Sample | Sample | PRESOL | Sample | Sample | Sample | Sample | PRESOL |
|---|---|---|---|---|---|---|---|---|---|---|---|---|---|
| | | A at | B at | C at | D at | E at | F at | at | C at | D at | E at | F at | at |
| | Control | 200 | 200 | 200 | 200 | 200 | 200 | 200 | 500 | 500 | 500 | 500 | 500 |
| Day | SFO | ppm | ppm | ppm | ppm | ppm | ppm | ppm | ppm | ppm | ppm | ppm | ppm |
| 0 | 0.758 | 0.758 | 0.758 | 0.758 | 0.758 | 0.758 | 0.758 | 0.758 | 0.758 | 0.758 | 0.758 | 0.758 | 0.758 |
| 15 | 4.368 | 2.008 | 3.636 | 3.551 | 4.285 | 3.446 | 3.618 | 2.687 | 4.232 | 4.164 | 3.945 | 4.38 | 2.426 |
| 30 | 13.706 | 2.927 | 8.816 | 5.794 | 8.638 | 5.292 | 6.656 | 3.478 | 6.862 | 8.44 | 6.027 | 6.985 | 3.096 |
| 45 | 19.876 | 3.223 | 10.813 | 8.277 | 9.698 | 7.17 | 8.878 | 4.076 | 10.058 | 9.205 | 8.654 | 10.858 | 3.541 |
| 60 | 27.018 | 3.335 | 14.091 | 10.01 | 13.774 | 9.427 | 12.059 | 4.687 | 12.682 | 13.552 | 11.797 | 14.07 | 4.007 |
| 75 | 32.633 | 3.711 | 17.632 | 11.944 | 16.788 | 11.138 | 13.25 | 5.269 | 13.733 | 15.923 | 12.073 | 14.205 | 4.353 |
| 90 | 39.991 | 4.088 | 22.615 | 13.127 | 21.797 | 12.531 | 14.842 | 6.462 | 16.117 | 20.813 | 13.58 | 17.56 | 4.8 |
| 105 | 48.713 | 4.77 | 28.884 | 17.58 | 26.33 | 15.304 | 19.278 | 7.312 | 19.456 | 25.293 | 16.844 | 20.159 | 5.486 |
| 120 | 57.501 | 5.13 | 33.526 | 22.001 | 28.554 | 20.025 | 24.212 | 8.328 | 23.059 | 27.932 | 20.989 | 25.356 | 6.176 |
| 135 | 67.881 | 5.809 | 41.168 | 25.038 | 35.363 | 23.046 | 28.144 | 9.478 | 25.936 | 34.289 | 23.77 | 29.161 | 6.838 |
| 150 | 77.186 | 6.387 | 45.546 | 30.001 | 40.412 | 27.58 | 33.795 | 10.037 | 31.597 | 39.268 | 28.875 | 35.537 | 7.648 |
| 165 | 86.946 | 7.875 | 55.612 | 35.109 | 51.02 | 33.144 | 37.494 | 11.674 | 36.1 | 49.919 | 34.131 | 39.538 | 8.613 |
| 180 | 92.913 | 8.252 | 60.578 | 39.591 | 54.15 | 37.852 | 42.26 | 12.461 | 40.22 | 52.295 | 38.821 | 44.094 | 9.462 |

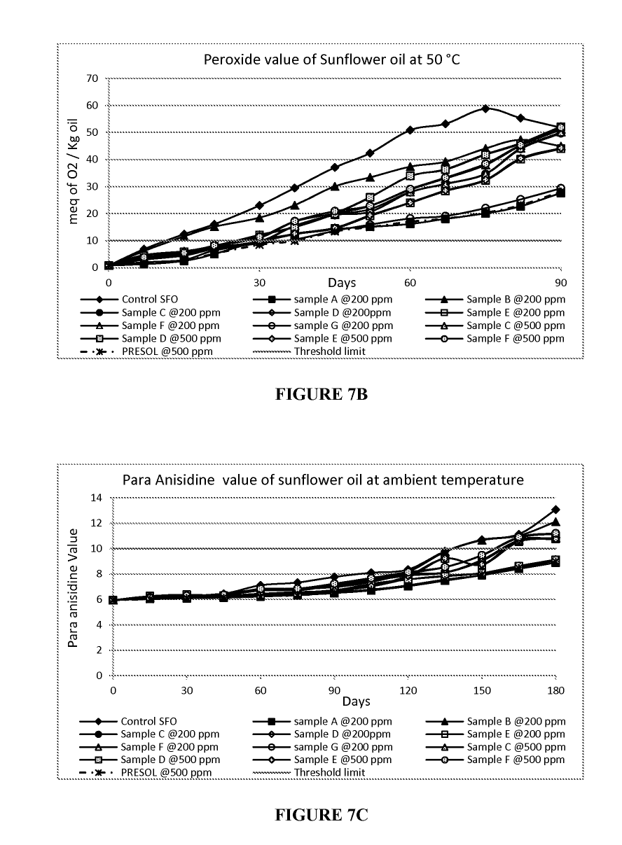

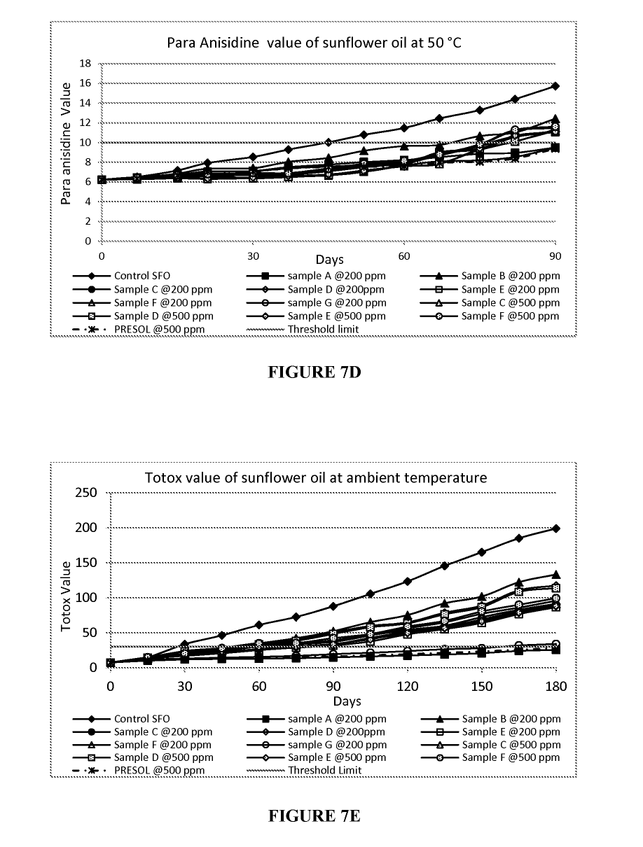

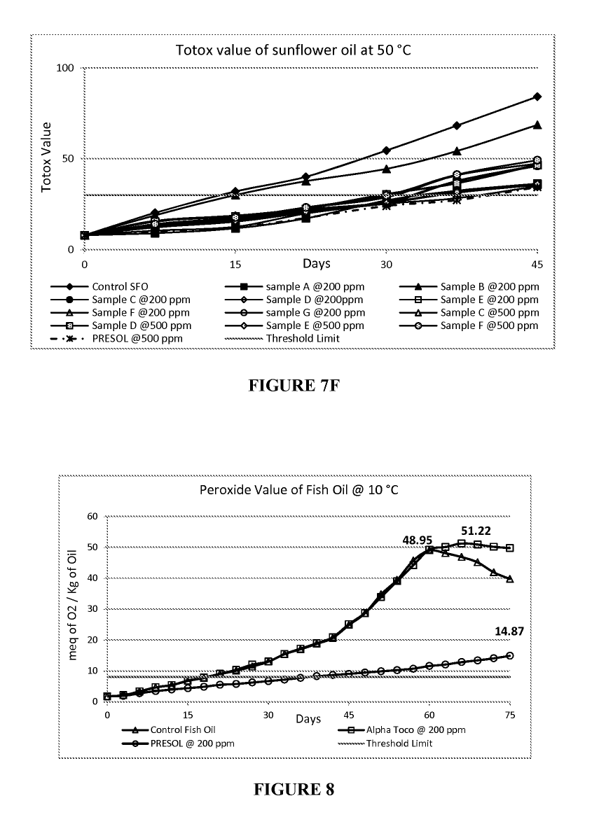

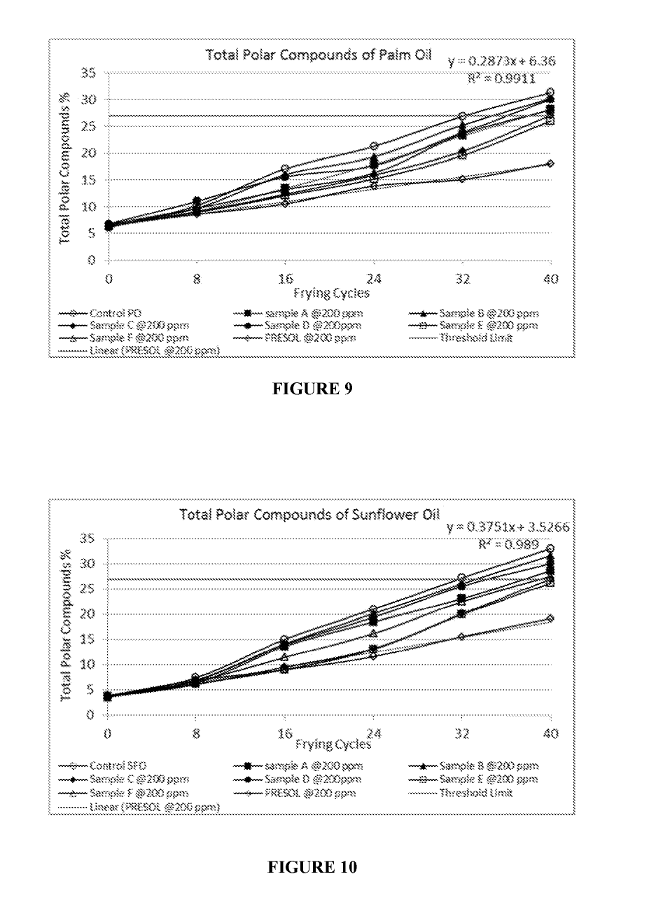

Table 14 and FIG. 7A illustrates that that PRESOL at 200 ppm and PRESOL at 500 ppm have better activity than Samples B, C, D, E and F. Control sample is observed to cross threshold limit within one month and other samples apart from PRESOL and sample A have crossed the threshold limit in two months. Further, PRESOL at 500 ppm is showing similar activity for preventive oxidative rancidity as compared to 200 ppm of TBHQ.

TABLE 15
Peroxide value of sunflower oil at 50° C.

| | | sample | Sample | Sample | Sample | Sample | Sample | PRESOL | Sample | Sample | Sample | Sample | PRESOL |
|---|---|---|---|---|---|---|---|---|---|---|---|---|---|
| | | A at | B at | C at | D at | E at | F at | at | C at | D at | E at | F at | at |
| | Control | 200 | 200 | 200 | 200 | 200 | 200 | 200 | 500 | 500 | 500 | 500 | 500 |
| Day | SFO | ppm | ppm | ppm | ppm | ppm | ppm | ppm | ppm | ppm | ppm | ppm | ppm |
| 0 | 0.807 | 0.807 | 0.807 | 0.807 | 0.807 | 0.807 | 0.807 | 0.807 | 0.807 | 0.807 | 0.807 | 0.807 | 0.807 |
| 7 | 6.862 | 1.293 | 6.172 | 3.01 | 4.663 | 2.964 | 3.615 | 2.01 | 3.388 | 4.458 | 3.161 | 3.805 | 1.607 |
| 15 | 12.413 | 2.49 | 11.596 | 4.483 | 6.079 | 4.456 | 5.065 | 2.892 | 4.89 | 5.865 | 4.645 | 5.472 | 2.631 |
| 21 | 16.064 | 5.051 | 15.146 | 7.447 | 8.043 | 7.022 | 8.066 | 6.387 | 7.67 | 7.905 | 7.2 | 8.199 | 5.183 |
| 30 | 23.001 | 9.989 | 18.486 | 9.431 | 12.125 | 9.556 | 11.001 | 9.053 | 9.722 | 12.034 | 9.656 | 11.413 | 8.456 |
| 37 | 29.478 | 12.472 | 23.087 | 15.431 | 15.066 | 12.375 | 17.042 | 10.425 | 15.522 | 14.949 | 12.584 | 17.172 | 9.91 |

Table 15 and FIG. 7B illustrates that all the samples apart from PRESOL is crossing the threshold limit within one month. Since sunflower oil is unstable at higher temperature and is more susceptible to oxidation at higher temperature, PRESOL is showing better activity in preventing oxidative rancidity of sunflower oil up to 37 days.

TABLE 16
Para-anisidine value of sunflower oil at ambient temperature

| | | sample | Sample | Sample | Sample | Sample | Sample | PRESOL | Sample | Sample | Sample | Sample | PRESOL |
|---|---|---|---|---|---|---|---|---|---|---|---|---|---|
| | | A at | B at | C at | D at | E at | F at | at | C at | D at | E at | F at | at |
| | Control | 200 | 200 | 200 | 200 | 200 | 200 | 200 | 500 | 500 | 500 | 500 | 500 |
| Day | SFO | ppm | ppm | ppm | ppm | ppm | ppm | ppm | ppm | ppm | ppm | ppm | ppm |
| 0 | 5.941 | 5.941 | 5.941 | 5.941 | 5.941 | 5.941 | 5.941 | 5.941 | 5.941 | 5.941 | 5.941 | 5.941 | 5.941 |
| 15 | 6.217 | 6.035 | 6.166 | 6.107 | 6.186 | 6.262 | 6.144 | 6.075 | 6.112 | 6.141 | 6.264 | 6.116 | 6.024 |
| 30 | 6.29 | 6.083 | 6.258 | 6.252 | 6.212 | 6.378 | 6.286 | 6.142 | 6.254 | 6.208 | 6.364 | 6.301 | 6.102 |
| 45 | 6.443 | 6.141 | 6.452 | 6.331 | 6.311 | 6.297 | 6.382 | 6.17 | 6.334 | 6.293 | 6.306 | 6.366 | 6.152 |
| 60 | 7.115 | 6.224 | 6.793 | 6.702 | 6.37 | 6.405 | 6.792 | 6.256 | 6.772 | 6.354 | 6.453 | 6.836 | 6.23 |
| 75 | 7.324 | 6.326 | 6.85 | 6.733 | 6.505 | 6.554 | 6.822 | 6.376 | 6.709 | 6.507 | 6.578 | 6.853 | 6.335 |
| 90 | 7.771 | 6.493 | 6.962 | 7.083 | 6.73 | 6.651 | 7.222 | 6.556 | 7.119 | 6.704 | 6.693 | 7.242 | 6.513 |
| 105 | 8.101 | 6.712 | 7.381 | 7.492 | 7.169 | 7.022 | 7.601 | 6.773 | 7.513 | 7.236 | 7.037 | 7.686 | 6.746 |
| 120 | 8.352 | 7.039 | 8.154 | 7.885 | 7.583 | 7.729 | 8.053 | 7.109 | 8.006 | 7.585 | 7.853 | 8.15 | 7.056 |
| 135 | 9.794 | 7.482 | 9.748 | 8.106 | 7.859 | 9.22 | 8.532 | 7.567 | 8.126 | 7.819 | 9.234 | 8.551 | 7.538 |
| 150 | 10.698 | 7.901 | 10.661 | 9.029 | 8.07 | 8.724 | 9.467 | 7.903 | 9.109 | 8.057 | 8.756 | 9.484 | 7.935 |
| 165 | 11.13 | 8.408 | 11.013 | 10.605 | 8.633 | 10.551 | 10.85 | 8.534 | 10.652 | 8.613 | 10.692 | 10.904 | 8.466 |
| 180 | 13.065 | 8.896 | 12.115 | 10.803 | 9.136 | 10.767 | 11.164 | 9.046 | 10.827 | 9.123 | 10.772 | 11.211 | 8.982 |

TABLE 17
Para-anisidine value of sunflower oil at 50° C.

| | | sample | Sample | Sample | Sample | Sample | Sample | PRESOL | Sample | Sample | Sample | Sample | PRESOL |
|---|---|---|---|---|---|---|---|---|---|---|---|---|---|
| | | A at | B at | C at | D at | E at | F at | at | C at | D at | E at | F at | at |
| | Control | 200 | 200 | 200 | 200 | 200 | 200 | 200 | 500 | 500 | 500 | 500 | 500 |
| Day | SFO | ppm | ppm | ppm | ppm | ppm | ppm | ppm | ppm | ppm | ppm | ppm | ppm |
| 0 | 6.237 | 6.237 | 6.237 | 6.237 | 6.237 | 6.237 | 6.237 | 6.237 | 6.237 | 6.237 | 6.237 | 6.237 | 6.237 |
| 7 | 6.511 | 6.334 | 6.439 | 6.435 | 6.483 | 6.262 | 6.374 | 6.384 | 6.453 | 6.444 | 6.285 | 6.403 | 6.348 |
| 15 | 7.165 | 6.675 | 6.842 | 6.548 | 6.51 | 6.378 | 6.636 | 6.706 | 6.555 | 6.499 | 6.401 | 6.689 | 6.681 |
| 21 | 7.926 | 7.026 | 7.347 | 6.623 | 6.608 | 6.297 | 6.742 | 7.068 | 6.688 | 6.54 | 6.338 | 6.805 | 7.05 |
| 30 | 8.532 | 7.11 | 7.392 | 6.702 | 6.37 | 6.405 | 6.729 | 7.091 | 6.82 | 6.36 | 6.467 | 6.923 | 7.063 |
| 37 | 9.28 | 7.503 | 8.035 | 6.733 | 6.505 | 6.554 | 6.792 | 7.383 | 6.781 | 6.492 | 6.62 | 6.897 | 7.383 |
| 45 | 10.014 | 7.754 | 8.438 | 7.083 | 6.73 | 6.651 | 7.222 | 7.558 | 7.255 | 6.707 | 6.756 | 7.372 | 7.504 |
| 52 | 10.775 | 8.016 | 9.162 | 7.492 | 7.169 | 7.022 | 7.698 | 7.697 | 7.509 | 7.149 | 7.104 | 7.841 | 7.538 |
| 60 | 11.464 | 8.208 | 9.641 | 7.885 | 7.583 | 7.729 | 8.112 | 7.718 | 7.964 | 7.593 | 7.761 | 8.132 | 7.597 |
| 67 | 12.435 | 8.506 | 9.748 | 8.693 | 7.859 | 8.924 | 8.85 | 8.097 | 8.724 | 7.813 | 9.014 | 8.793 | 7.949 |
| 75 | 13.278 | 8.863 | 10.661 | 9.613 | 9.249 | 9.313 | 9.763 | 8.182 | 9.696 | 9.328 | 9.386 | 9.815 | 8.06 |
| 82 | 14.388 | 8.945 | 11.013 | 10.605 | 10.109 | 10.551 | 11.195 | 8.526 | 10.63 | 10.084 | 10.692 | 11.275 | 8.38 |
| 90 | 15.714 | 9.484 | 12.411 | 11.098 | 11.188 | 11.061 | 11.462 | 9.471 | 11.146 | 11.319 | 11.059 | 11.611 | 9.378 |

Table 16, table 17 and FIG. 7C illustrates that PRESOL is having better activity when compared to oils of samples B, C, D, E and F and it is having activity similar to oil comprising TBHQ.

TABLE 18
Totox value of sunflower oil at ambient temperature

| | | sample | Sample | Sample | Sample | Sample | Sample | PRESOL | Sample | Sample | Sample | Sample | PRESOL |
|---|---|---|---|---|---|---|---|---|---|---|---|---|---|
| | | A at | B at | C at | D at | E at | F at | at | C at | D at | E at | F at | at |
| | Control | 200 | 200 | 200 | 200 | 200 | 200 | 200 | 500 | 500 | 500 | 500 | 500 |
| Day | SFO | ppm | ppm | ppm | ppm | ppm | ppm | ppm | ppm | ppm | ppm | ppm | ppm |
| 0 | 7.458 | 7.458 | 7.458 | 7.458 | 7.458 | 7.458 | 7.458 | 7.458 | 7.458 | 7.458 | 7.458 | 7.458 | 7.458 |
| 15 | 14.953 | 10.051 | 13.438 | 13.210 | 14.756 | 13.154 | 13.380 | 11.449 | 14.577 | 14.469 | 14.153 | 14.877 | 10.877 |
| 30 | 33.701 | 11.937 | 23.889 | 17.839 | 23.488 | 16.962 | 19.598 | 13.098 | 19.977 | 23.089 | 18.417 | 20.270 | 12.294 |
| 45 | 46.195 | 12.588 | 28.077 | 22.885 | 25.708 | 20.637 | 24.138 | 14.323 | 26.450 | 24.702 | 23.615 | 28.082 | 13.234 |
| 60 | 61.152 | 12.893 | 34.976 | 26.722 | 33.918 | 25.258 | 30.909 | 15.629 | 32.137 | 33.457 | 30.046 | 34.977 | 14.243 |
| 75 | 72.589 | 13.749 | 42.114 | 30.622 | 40.080 | 28.831 | 33.322 | 16.914 | 34.175 | 38.353 | 30.725 | 35.262 | 15.042 |
| 90 | 87.753 | 14.669 | 52.191 | 33.337 | 50.324 | 31.714 | 36.906 | 19.479 | 39.354 | 48.329 | 33.854 | 42.363 | 16.113 |
| 105 | 105.528 | 16.253 | 65.149 | 42.652 | 59.829 | 37.629 | 46.156 | 21.396 | 46.424 | 57.823 | 40.725 | 48.004 | 17.717 |
| 120 | 123.353 | 17.299 | 75.207 | 51.888 | 64.690 | 47.780 | 56.477 | 23.765 | 54.124 | 63.450 | 49.832 | 58.862 | 19.408 |
| 135 | 145.557 | 19.100 | 92.085 | 58.183 | 78.584 | 55.311 | 64.820 | 26.522 | 59.997 | 76.398 | 56.775 | 66.872 | 21.215 |
| 150 | 165.070 | 20.674 | 101.75 | 69.030 | 88.894 | 63.884 | 77.056 | 27.976 | 72.303 | 86.593 | 66.505 | 80.558 | 23.230 |
| 165 | 185.022 | 24.158 | 122.23 | 80.822 | 110.674 | 76.838 | 85.838 | 31.882 | 82.851 | 108.45 | 78.953 | 89.981 | 25.692 |
| 180 | 198.890 | 25.399 | 133.27 | 89.984 | 117.436 | 86.471 | 95.684 | 33.968 | 91.268 | 113.71 | 88.413 | 99.400 | 27.906 |

TABLE 19
Totox value of sunflower oil at 50° C.

| | | sample | Sample | Sample | Sample | Sample | Sample | PRESOL | Sample | Sample | Sample | Sample | PRESOL |
|---|---|---|---|---|---|---|---|---|---|---|---|---|---|
| | | A at | B at | C at | D at | E at | F at | at | C at | D at | E at | F at | at |
| | Control | 200 | 200 | 200 | 200 | 200 | 200 | 200 | 500 | 500 | 500 | 500 | 500 |
| Day | SFO | ppm | ppm | ppm | ppm | ppm | ppm | ppm | ppm | ppm | ppm | ppm | ppm |
| 0 | 7.850 | 7.850 | 7.850 | 7.850 | 7.850 | 7.850 | 7.850 | 7.850 | 7.850 | 7.850 | 7.850 | 7.850 | 7.850 |
| 7 | 20.235 | 8.919 | 18.784 | 12.456 | 15.810 | 12.190 | 13.603 | 10.403 | 13.229 | 15.359 | 12.607 | 14.013 | 9.563 |
| 15 | 31.991 | 11.655 | 30.034 | 15.515 | 18.669 | 15.291 | 16.765 | 12.490 | 16.335 | 18.229 | 15.692 | 17.633 | 11.944 |
| 22 | 40.055 | 17.128 | 37.639 | 21.518 | 22.695 | 20.341 | 22.873 | 19.842 | 22.028 | 22.350 | 20.738 | 23.203 | 17.416 |
| 30 | 54.533 | 27.087 | 44.365 | 25.564 | 30.620 | 25.517 | 28.731 | 25.197 | 26.265 | 30.428 | 25.780 | 29.748 | 23.975 |
| 37 | 68.235 | 32.447 | 54.210 | 37.595 | 36.637 | 31.304 | 40.876 | 28.234 | 37.826 | 36.390 | 31.789 | 41.240 | 27.204 |
| 45 | 84.177 | 36.287 | 68.642 | 46.012 | 46.960 | 35.568 | 47.348 | 34.882 | 46.492 | 46.600 | 36.207 | 49.221 | 34.344 |

Table 18 and FIG. 7E illustrates that 200 ppm and 500 ppm of PRESOL is having better activity than antioxidant in samples B, C, D, E and F. Further, control is observed to cross threshold limit within one month. 200 ppm PRESOL is observed to prevent oxidative rancidity of sunflower oil up to 165 days and 500 ppm PRESOL is observed to prevent oxidative rancidity of sunflower oil up to 180 days. From this illustration, it can inferred that PRESOL concentration can be increased accordingly in order to prevent oxidative rancidity of oils, whereas the concentration of synthetic antioxidants cannot be increased over a particular limit as per the regulatory guidelines as they might cause harmful effects. For instance, TBHQ is a synthetic antioxidant, which cannot be used beyond the concentration of 200 ppm, as it is considered to be carcinogenic beyond 200 ppm. Further form the table 19 and FIG. 7F, it can be observed that the totox value of sunflower oil with PRESOL (200 ppm and 500 ppm) does not cross the threshold limit of 30 till 37 days, whereas sunflower oil with all other antioxidants (samples A, B, C, D, E, F) crosses the threshold limit by 37 days.

C. Determination of Shelf Life of Fish Oil

TABLE 20
Peroxide value of fish oil at 10° C.

| | | Alpha Toco | |
|---|---|---|---|
| | Control | (Sample A1) | PRESOL |
| Day | Fish Oil | at 200 ppm | at 200 ppm |
| 0 | 1.74 | 1.74 | 1.74 |
| 3 | 2.09 | 2.13 | 1.89 |
| 6 | 3.26 | 3.33 | 2.73 |
| 9 | 4.62 | 4.71 | 3.46 |
| 12 | 5.39 | 5.41 | 3.97 |
| 15 | 6.74 | 6.82 | 4.39 |
| 18 | 7.69 | 7.84 | 4.85 |
| 21 | 8.98 | 9.02 | 5.48 |
| 24 | 10.06 | 10.32 | 5.74 |
| 27 | 11.38 | 12.02 | 6.24 |
| 30 | 12.97 | 13.01 | 6.68 |
| 33 | 15.39 | 15.44 | 7.14 |
| 36 | 16.98 | 17.16 | 7.72 |
| 39 | 18.72 | 18.94 | 8.26 |
| 42 | 20.63 | 20.86 | 8.63 |
| 45 | 24.82 | 25.09 | 9.01 |
| 48 | 28.63 | 28.71 | 9.45 |
| 51 | 34.74 | 33.89 | 9.87 |
| 54 | 39.42 | 39.01 | 10.23 |
| 57 | 44.66 | 44.14 | 10.69 |
| 60 | 48.95 | 49.22 | 11.57 |
| 63 | 48.05 | 50.06 | 12.03 |
| 66 | 46.82 | 51.22 | 12.84 |
| 69 | 45.08 | 50.85 | 13.38 |
| 72 | 41.86 | 50.13 | 14.05 |
| 75 | 39.71 | 49.72 | 14.87 |

Table 20 and FIG. 8 illustrates that peroxide value of control fish oil and fish oil added with alpha tocopherol (200 ppm) crosses the threshold limit of 8 meq/kg after 21 days, whereas fish oil with 500 ppm of PRESOL is does not cross the threshold limit till 39 days of storage, hence, proving PRESOL to be more powerful antioxidant than synthetic antioxidants.

D. Determination of Shelf Life of Canola Oil in the Presence of PRESOL
Shelf life of canola oil is analyzed upon adding 200 ppm of TBHQ (sample-A), BHA (sample-B), green tea extract with 90% polyphenol (sample-C), rosemary extract (sample-D), green tea extract after cryogenic grinding (sample-E), and PRESOL, respectively to canola oil.

TABLE 21

| Sample | Shelf Life (No. Of Days) |
|---|---|
| Control (Canola Oil) | 58 |
| Sample-A | 181 |
| Sample-B | 37 |
| Sample-C | 149 |
| Sample-D | 74 |
| Sample-E | 202 |
| PRESOL | 256 |

From the table 21, it is illustrated that PRESOL increases the shelf life of canola oil by enhancing oxidative stability of the oil.

### Example 4

### Frying Cycle Analysis

Palm oil and sunflower oil with antioxidants added to it are obtained from a local refinery. Frying studies are carried out with control oil and by adding TBHQ (Sample A), BHA (Sample B), GT Pulverized (Sample C), ROS (Sample D), GT Cryo grinded (Sample E), GT Crude Extract (Sample F) and PRESOL, respectively to the oils. Experiments are carried out a concentration of 200 ppm and 500 ppm. Fresh potatoes of less reduced sugar content variety are used throughout the experiment which is purchased form a local supermarket. Oil samples are withdrawn from the fryer at the end of every day and stored at −4° C. until it is tested for quality parameters viz, total polar compounds. Polar compounds present in oil and fats are measured by column chromatography using standard method, ES ISO 8420:2012.

A. Frying Cycle Analysis of Palm Oil

TABLE 22
Total polar compounds of Palm oil

| | | sample | Sample | Sample | Sample | Sample | Sample | PRESOL | |
|---|---|---|---|---|---|---|---|---|---|
| | | A at | B at | C at | D at | E at | F at | at | |
| | Control | 200 | 200 | 200 | 200 | 200 | 200 | 200 | Threshold |
| Cycle | PO | ppm | ppm | ppm | ppm | ppm | ppm | ppm | Limit |
| 0 | 6.057 | 6.307 | 6.325 | 6.357 | 6.752 | 6.304 | 6.655 | 6.528 | 27 |
| 8 | 10.275 | 9.18 | 9.754 | 9.087 | 11.039 | 8.944 | 9.703 | 8.564 | 27 |
| 16 | 17.083 | 13.4 | 15.964 | 12.322 | 15.534 | 12.071 | 13.17 | 10.565 | 27 |
| 24 | 21.268 | 17.913 | 19.329 | 15.872 | 17.699 | 15.127 | 16.264 | 13.855 | 27 |
| 32 | 26.9 | 23.217 | 25.264 | 20.351 | 23.851 | 19.55 | 23.583 | 15.064 | 27 |
| 40 | 31.33 | 28.187 | 30.192 | 27.059 | 30.051 | 26.005 | 28.072 | 18.058 | 27 |

Table 22 illustrates that palm oil with TBHQ, BHA, GT Pulverized, ROS, GT Cryo grinded and GT Crude Extract have crossed threshold limit for total polar compounds after 40^{th} cycle except oil with PRESOL.

B. Frying Cycle Analysis of Sunflower Oil

TABLE 23
Total Polar compounds of sunflower oil

| | | sample | Sample | Sample | Sample | Sample | Sample | PRESOL | |
|---|---|---|---|---|---|---|---|---|---|
| | | A at | B at | C at | D at | E at | F at | at | |
| | Control | 200 | 200 | 200 | 200 | 200 | 200 | 200 | Threshold |
| Cycle | SFO | ppm | ppm | ppm | ppm | ppm | ppm | ppm | Limit |
| 0 | 3.616 | 3.746 | 3.515 | 3.497 | 3.872 | 3.763 | 3.763 | 3.856 | 27 |
| 8 | 7.45 | 6.375 | 6.958 | 6.079 | 6.888 | 6.244 | 6.742 | 6.938 | 27 |
| 16 | 15.037 | 13.632 | 14.124 | 9.548 | 13.986 | 9.048 | 11.47 | 9.023 | 27 |
| 24 | 21.025 | 18.505 | 20.105 | 13.17 | 19.39 | 13.062 | 16.181 | 11.616 | 27 |
| 32 | 27.194 | 23.123 | 26.085 | 20.137 | 25.589 | 19.98 | 22.511 | 15.584 | 27 |
| 40 | 33.079 | 28.648 | 31.692 | 27.00 | 30.073 | 26.182 | 27.574 | 19.156 | 27 |

Table 23 illustrates that sunflower oil with TBHQ, BHA, GT Pulverized, ROS, GT Cryo grinded and GT Crude Extract have crossed threshold limit for total polar compounds after 40^{th} cycle except oil with PRESOL.

### Example 4

### Effect of Particle Size on Antioxidant Activity of Vegetable Oils

Effect of particle size analysis on antioxidant activity is analysed with the help of Rancimat (Metrohm 743) which gives oxidative stability index based on the induction time (h). Rancimat analysis is carried out at two different temperature in order to set relationship between induction time and temperature of the studies.

Table 24 shows the particle size of different samples and its effect on antioxidant activity. In this, we have analyzed the particle size of samples with the help of Particle Size Analyzer (Microtrac, S3500) and its antioxidant activity with the help of Rancimat (Metrohm 743). Table 24 shows that reducing the particle size of GT extract has an impact on antioxidant activity. It is observed that when the particle size of GT is reduced, the Rancimat induction time increases which proves that the antioxidant activity increases on reduction of particle size. Further, the optimum particle size is between 5 to 10 μm at which the maximum activity is achieved.

TABLE 24
Relationship between particle size and antioxidant
activity of TBHQ (sample-A), 200 ppm of BHA (sample-B),
200 ppm of pulverized green tea extract with 90% polyphenol
(sample-C), 200 ppm of rosemary extract (sample-D),
200 ppm of green tea extract after cryogenic
grinding (sample-E), 200 ppm of crude green tea
extract and PRESOL at 200 ppm, 500 ppm and 1000 ppmn
in palm oil (PO) and sunflower oil (SFO), respectively.

| | | Induction | Induction |
|---|---|---|---|
| | Particle | Period (h) of | Period (h) of |
| Treatment | Size (μm) | PO at 120° C. | SFO 100° C. |
| Control | | 9.64 | 9.32 |
| Sample A | Crystalline | 20.93 | 26.82 |
| | form | | |
| Sample B | Crystalline | 9.88 | 13.33 |
| | form | | |
| Sample C | 46.48 | 17.95 | 24.03 |
| Sample D | Dispersed | 13.32 | 12.94 |
| | form | | |
| Sample E | 12.81 | 20.02 | 25.28 |
| Sample F | 119.3 | 14.17 | 19.08 |
| PRESOL | 6.12 | 15.57 | 18.38 |
| PRESOL | 6.12 | 23.74 | 22.76 |
| PRESOL | 6.12 | 33.64 | 34.41 |

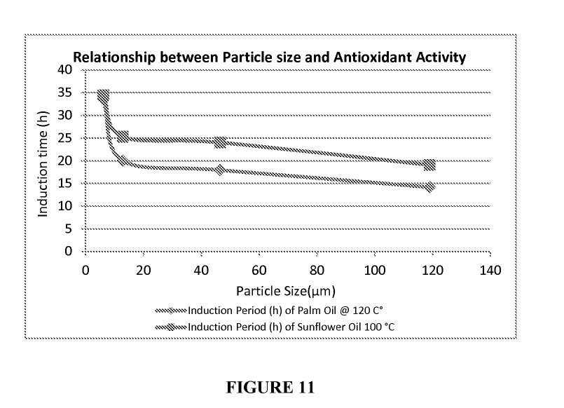

From table 24 and FIG. 11 it is illustrated that reducing particle size increases the solubility and aids to prevent sedimentation of the active components in oil matrix. As observed in the table, PRESOL at lower particle size is able to show better antioxidant activity thereby reducing the oxidative rancidity of the oil.

### Example 6

### Fortification of Tertiary Butyl Hydroquinone (TBHQ) and PRESOL

Synergism of TBHQ and PRESOL is analyzed in palm oil. If the frying industry is considered, synthetic antioxidant TBHQ is being added to the oils. However, there is a limitation of adding TBHQ i.e. not above 200 ppm, due to which frying oils cannot be used for longer frying cycles. In order to overcome this limitation, TBHQ is fortified with PRESOL and added to vegetable oils in order to increase the frying cycles which ultimately increases the shelf life of end products. Fortification of TBHQ & PRESOL is analyzed in Rancimat at different concentrations, ranging from about 50 to 200 ppm in palm oil. The below table 25 illustrates all the combination of fortification of TBHQ and PRESOL used in palm oil to illustrate maximum activity of fortified combination.

Synergism is calculated based on the below equation:

$$Synergism ( \% ) = \frac{( {IP}_{X 1 X 2} - {IP}_{o} ) - [ ( {IP}_{X 1} - {IP}_{o} ) + ( {IP}_{X 2} - {IP}_{o} ) ]}{[ ( {IP}_{X 1} - {IP}_{o} ) + ( {IPIP}_{X 2} - {IP}_{o} ) ]} \times 100$$

### Where,

### IP₀=Induction Period for Control Oil

### IP_{X1}=Induction Period for X1 (TBHQ)

### IP_{X2}=Induction Period for X2 (PRESOL)

IP_{X1x2}=Induction Period for combination of X1 (TBHQ) and X2 (PRESOL)

TABLE 25
Synergism of TBHQ and PRESOL combination.

| | TBHQ | PRESOL | Induction | increase in | | | |
|---|---|---|---|---|---|---|---|
| | (Sample | (Sample | Time (H) | induction time | | Increase | |
| Vegetable | A)(ppm) | G)(ppm) | at | as compared to | | in OSI | Synergism |
| Oil | X1 | X2 | 120 C. | control (X-C) | OSI | (%) | (%) |
| PO | 0 | 0 | 9.08 | 0.00 | 1 | | |
| Control | | | | | | | |
| (C) | | | | | | | |
| PO | 50 | 0 | 9.57 | 0.49 | 1.054 | 5.12 | |
| PO | 100 | 0 | 11.01 | 1.93 | 1.21 | 17.53 | |
| PO | 200 | 0 | 14.24 | 5.16 | 1.57 | 36.24 | |
| PO | 0 | 50 | 10.52 | 1.44 | 1.16 | 13.69 | |
| PO | 0 | 100 | 11.36 | 2.28 | 1.25 | 20.07 | |
| PO | 0 | 200 | 15.57 | 6.49 | 1.71 | 41.68 | |
| PO | 200 | 100 | 31.8 | 22.72 | 3.50 | 71.45 | 205.38 |
| PO | 100 | 200 | 25.71 | 16.63 | 2.83 | 64.68 | 97.51 |
| PO | 100 | 100 | 30.05 | 20.97 | 3.31 | 69.78 | 398.10 |
| PO | 50 | 50 | 11.63 | 2.55 | 1.28 | 21.93 | 32.12 |
| PO | 200 | 200 | 29.32 | 20.24 | 3.23 | 69.03 | 73.73 |
| PO | 50 | 100 | 20.34 | 11.26 | 2.24 | 55.35 | 306.50 |
| PO | 50 | 200 | 24.09 | 15.01 | 2.65 | 62.31 | 115.04 |
| PO | 100 | 50 | 18.5 | 9.42 | 2.04 | 50.92 | 179.53 |
| PO | 200 | 50 | 22.44 | 13.36 | 2.47 | 59.54 | 102.42 |

Table 25 illustrates that the combination of 100 ppm of TBHQ and 100 ppm of PRESOL is showing synergistic antioxidant activity by suitably enhancing the oxidative stability of palm oil.

### Example 7

### Solubility of PRESOL in Sunflower Oil

Solubility of PRESOL is tested with respect to pulverized green tea extract (sample-C), green tea extract cryogrinded (sample-E) and crude green tea (sample-F) in sunflower oil. Solubility test is carried out by analyzing the settling percentage in the oil over storage. Settling is determined by analyzing the polyphenol content, which is present in the oil comprising the above mentioned samples using spectrophotometer at 540 nm.

TABLE 26
Solubility of PRESOL in sunflower oil.

| | | | | Concentration | | |
|---|---|---|---|---|---|---|
| | | Concentration | Concentration | of Polyphenols | | |
| Trial | | of Blends in | Polyphenols | in Oil during | % | % |
| No | Treatments | ppm | in blends ppm | storage(ppm) | Solubility | Settling |
| 1 | PRESOL | 200 | 41.86 | 41.35 | 98.78 | 1.22 |
| 2 | | 500 | 104.65 | 102.54 | 97.98 | 2.02 |
| 3 | | 1000 | 209.3 | 200.85 | 95.96 | 4.04 |
| 4 | Sample C | 200 | 190 | 68.59 | 36.1 | 63.9 |
| 5 | | 500 | 475 | 147.96 | 31.15 | 68.85 |
| 6 | | 1000 | 950 | 253.175 | 26.65 | 73.35 |
| 7 | Sample E | 200 | 190 | 80.199 | 42.21 | 57.79 |
| 8 | | 500 | 475 | 179.55 | 37.8 | 62.2 |
| 9 | | 1000 | 950 | 314.45 | 33.1 | 66.9 |
| 10 | Sample F | 200 | 190 | 48.64 | 25.6 | 74.4 |
| 11 | | 500 | 475 | 85.56 | 18.01 | 81.99 |
| 12 | | 1000 | 950 | 99.37 | 10.46 | 89.54 |

$$Total Polyphenols \% = \frac{Wt . of std \times 10 \times Abs . of sample \times Ds \times P \times 100}{100 \; 50 Abs . of std}$$

### Where,

Ds=dilution of sample
Wt=Weight of the sample in gram
P=Purity of standard

From the table 26, it is illustrated that settling is on the higher side for green tea extract without grinding (sample-F) followed by pulverized green tea extract (sample-C) and at a lower side for green tea extract with cryogrinding (sample-E). However, PRESOL is having highest solubility percentage in the oil with very minimal settling over storage. The high solubility of PRESOl with minimal settling is directly linked to the percentage of polyphenols present in the composition.

Though Polyphenols is the active component which provides the antioxidant activity in green tea extracts, it has very poor solubility in oil. On the other hand, PRESOL with polyphenol percentage of about 30% to about 38% shows enhanced antioxidant activity with minimal settling over storage.

### Example 8

### Determining Solubility of PRESOL with Different Emulsifying Agents

Table 27 illustrates that PRESOL comprising Macrogoglycerol hydroxystearate is having an enhanced solubility with a percentage solubility ranging from about 95% to about 100%, with settling less than 5%

TABLE 27
Solubility of PRESOL comprising emulsifying agents

| | | | | | Concentration | | |
|---|---|---|---|---|---|---|---|
| | | | Concentration | Concentration | of Polyphenols | | |
| Trial | | Emulsifying | of Blends in | Polyphenols | in Oil during | % | % |
| No | Treatments | Agent | ppm | in blends ppm | storage(ppm) | Solubility | Settling |
| 1 | PRESOL | Macrogolglycerol | 200 | 41.86 | 41.35 | 98.78 | 1.22 |
| 2 | | Hydroxystearate | 500 | 104.65 | 102.54 | 97.98 | 2.02 |
| 3 | | | 1000 | 209.3 | 200.85 | 95.96 | 4.04 |
| 4 | PRESOL | Sorbitol (glucitol) | 200 | 41.86 | 27.26 | 65.11 | 34.89 |
| 5 | | | 500 | 104.65 | 66.02 | 63.09 | 36.91 |
| 6 | | | 1000 | 209.3 | 125.33 | 59.88 | 40.12 |
| 7 | PRESOL | DATEM | 200 | 41.86 | 33.17 | 79.25 | 20.75 |
| 8 | | (diacetyl tartaric | 500 | 104.65 | 80.01 | 76.45 | 23.55 |
| 9 | | acid ester of mono- | 1000 | 209.3 | 146.68 | 70.08 | 29.92 |
| | | and diglycerides) | | | | | |
| 10 | PRESOL | Guar gum | 200 | 41.86 | 25.76 | 61.53 | 38.47 |
| 11 | | (Galactomannan) | 500 | 104.65 | 60.94 | 58.23 | 41.77 |
| 12 | | | 1000 | 209.3 | 114.38 | 54.65 | 45.35 |

### Example 9

### Cost Advantage of PRESOL Over TBHQ

Case 9.1 with 200 Ppm TBHQ in Sunflower Oil (SFO)

Consider a fryer with oil bath capacity of 100 litres. We are using SFO as our medium of oil for frying potato chips. Rate of oil is 90− per kg. Rate of TBHQ is approx. 750− per kg. TBHQ dosage is 200 ppm, which is 0.2 gms in 1000 gms of oil i.e. 0.15 rupees per kg. So the net rate of oil will be (90+1.5)=90.15− per kg. Number of batches fried after adding 200 ppm TBHQ is 40 batches. Hence, total cost incurred per batch of frying will be, 100*90.15/40=225.375− per batch.

Case 9.2 with 200 Ppm PRESOL in Sunflower Oil (SFO)

Consider a fryer with oil bath capacity of 100 litres. We are using SFO as our medium of oil for frying potato chips. Rate of oil is 90− per kg. Rate of PRESOL is approx. 2500− per kg. PRESOL dosage is 200 ppm, which is 0.2 gms in 1000 gms of oil i.e. 0.5 rupees per kg. So the net rate of oil will be (90+0.5)=90.5− per kg. Number of batches fried after adding 200 ppm PRESOL is 62 batches. Hence, total cost incurred per batch of frying will be, 100*90.5/62=146.97− per batch.

Cost saved is about rupees78− (Approx. 35% reduction per Batch).
Case 9.3 with 200 Ppm TBHQ in Palm Oil (PO)

Consider a fryer with oil bath capacity of 100 litres. We are using PO as our medium of oil for frying potato chips. Rate of oil is 50− per kg. Rate of TBHQ is approx. 750− per kg. TBHQ dosage is 200 ppm, which is 0.2 gms in 1000 gms of oil i.e. 0.15 rupees per kg. So the net rate of oil will be (50+0.15)=50.15− per kg. Number of batches fried after adding 200 ppm TBHQ is 40 batches. Hence, total cost incurred per batch of frying will be, 100*50.15/40=125.375− per batch.

Case 9.4 with 200 Ppm PRESOL in Palm Oil (PO)

Consider a fryer with oil bath capacity of 100 litres. We are using PO as our medium of oil for frying potato chips. Rate of oil is 50− per kg. Rate of PRESOL is approx. 2500− per kg. PRESOL dosage is 200 ppm, which is 0.2 gms in 1000 gms of oil i.e. 0.5 rupees per kg. So the net rate of oil will be (50+5)=50.5− per kg. Number of batches fried after adding 200 ppm PRESOL is 72 batches. Hence, total cost incurred per batch of frying will be, 100*50.5/72=70.14− per batch.

Cost saved is about rupees 55− (Approx. 44% reduction per Batch).

Although cost is a relative parameter which changes with time, the costs showcased herein are applicable in August 2013, and are presented to provide an idea of the cost differential between the composition of the instant invention and the most used sample, TBHQ. This relative differential will remain the same

1. A composition comprising green tea extract and rosemary extract, optionally along with adjuvant or excipient or a combination thereof.

2. The composition as claimed in claim 1, wherein said green tea extract is at a concentration ranging from about 24% w/w to about 80% w/w.

3. The composition as claimed in claim 1, wherein said rosemary extract is at a concentration ranging from about 45% w/w to about 85% w/w.

4. The composition as claimed in claim 1, wherein said rosemary extract comprises rosemarinic acid at a concentration ranging from about 0.5% w/w to about 10% w/w and carnosic acid at a concentration ranging from about 1.0% w/w to about 20% w/w.

5. The composition as claimed in claim 1, wherein the adjuvant is selected from a group comprising vitamin C, gallic acid, vitamin E, rosmanol, ferulic acid, citric acid, mixed-tocopherol, lecithin, carotenoids and uric acid or any combination thereof.

6. The composition as claimed in claim 1, wherein the excipient is selected from a group comprising granulating agent, binding agent, lubricating agent, disintegrating agent, sweetening agent, glidant, anti-adherent, anti-static agent, surfactant, coating agent, colouring agent, flavouring agent, plasticizer, suspending agent, additive, emulsifying agent and spheronization agent or any combination thereof.

7. The composition as claimed in claim 6, wherein the emulsifying agent is polyglyceride fatty acid ester; and wherein the additive is selected from a group comprising mono-di-glycerides, sorbitol, guar gum and xanthan gum or any combination thereof

8. The composition as claimed in claim 5, wherein said adjuvant is at a concentration ranging from about 1.0 w/w to about 10% w/w.

9. The composition as claimed in claim 6, wherein said excipient is at a concentration ranging from about 0.5% to about 5% w/w.

10. The composition as claimed in claim 1, wherein particle size of the composition ranges from about 5 μm to about 10 μm.

11. The composition as claimed in claim 1, wherein the composition optionally comprises polyphenols at a concentration ranging from about 30% to about 38%.

12. The composition as claimed in claim 1 is oil soluble having solubility ranging from about 95% to about 100% with settling less than about 0 about 5%.

13. A process of preparing the composition comprising green tea extract and rosemary extract, said process comprising acts of:
a. granulating crude extract of green tea and mixing the granulated extract with the rosemary extract to obtain a mixture;
b. optionally adding adjuvant or excipient or a combination thereof to the mixture; and
c. passing the mixture of step a) or b) through homogenizer to obtain said composition comprising green tea extract and rosemary extract

14. The process as claimed in claim 13, wherein the crude extract of green tea having a particle size of about 50μ to about 200μ is granulated to obtain a extract of particle size ranging from about 10μ to about 20μ.

15. The process as claimed in claim 13, wherein the composition obtained in step c) has a particle size ranging from about 5μ to about 10μ.

16. The process as claimed in claim 13, wherein the crude extract of the green tea is granulated by techniques selected from a group comprising hammer mill, ball mill, plate mill, disc mill, colloid mill, micronisation method, high pressure homogenization and cryogenic grinding or a combination thereof, preferably cryogenic grinding.

17. The process as claimed in claim 13, wherein the mixing is carried out by an agitator at a speed of about 500 rpm to about 1000 rpm.

18. The process as claimed in claim 17, wherein the mixing is at a temperature ranging from about 40° C. to about 65° C. for time period ranging from about 2 hrs to about 24 hrs.

19. The process as claimed in claim 13, wherein the mixture is homogenized at a pressure ranging from about 500 bar to about 1000 bar.

20. A composition comprising the composition as claimed in claim 1 and tertiary butyl hydroquinone.

21. The composition as claimed in claim 20, wherein the composition of claim 1 is at a concentration ranging from about 50 ppm to about 100 ppm.

22. The composition as claimed in claim 20, wherein the tertiary butyl hydroquinone is at a concentration ranging from about 50 ppm to about 100 ppm.

23. An oil, fat or cosmetic preparation, comprising the composition of claim 1.

24. A method of preparing the oil, fat or cosmetic preparation as claimed in claim 23, wherein said method comprises act of mixing the composition with the oil, fat or cosmetic ingredients.
